(19) United States
(12) Patent Application Publication
(10) Pub. No.: US 2013/0303559 A1
(43) Pub. Date: Nov. 14, 2013

(54) BENZIMIDAZOLE AND PYRAZOLOPYRIDINE DERIVATIVES FOR TREATING AND/OR PREVENTING CARDIOVASCULAR DISEASES

(71) Applicant: BAYER INTELLECTUAL PROPERTY GmbH, Monheim (DE)

(72) Inventors: Alexander STRAUB, Wuppertal (DE); Frank SÜßMEIER, Munchen (DE); Frank WUNDER, Wuppertal (DE); Johannes-Peter STASCH, Solingen (DE); Volkhart Min-Jian LI, Velbert (DE); Joachim MITTENDORF, Wuppertal (DE)

(21) Appl. No.: 13/941,967

(22) Filed: Jul. 15, 2013

Related U.S. Application Data
(63) Continuation of application No. 13/143,415, filed on Sep. 16, 2011, now abandoned.

(30) Foreign Application Priority Data
Jan. 9, 2009 (DE) 102009004245.8

(51) Int. Cl. C07D 403/04; A61K 31/506; A61K 45/06; C07D 471/04

(52) U.S. Cl. CPC C07D 403/04; C07D 471/04; A61K 31/506; A61K 45/06
USPC 514/275; 544/324

(57) ABSTRACT

The present application relates to novel fused, heteroatom-bridged pyrazole and imidazole derivatives, to processes for their preparation, to their use, alone or in combination, for the treatment and/or prevention of diseases and to their use for preparing medicaments for the treatment and/or prevention of diseases, in particular for the treatment and/or prevention of cardiovascular disorders.

The present application relates to novel fused, heteroatom-bridged pyrazole and imidazole derivatives, to processes for their preparation, to their use, alone or in combination, for the treatment and/or prevention of diseases and to their use for preparing medicaments for the treatment and/or prevention of diseases, in particular for the treatment and/or prevention of cardiovascular disorders.

One of the most important cellular transmission systems in mammalian cells is cyclic guanosine monophosphate (cGMP). Together with nitric oxide (NO), which is released from the endothelium and transmits hormonal and mechanical signals, it forms the NO/cGMP system. Guanylate cyclases catalyze the biosynthesis of cGMP from guanosine triphosphate (GTP). The representatives of this family disclosed to date can be divided both according to structural features and according to the type of ligands into two groups: the particulate guanylate cyclases which can be stimulated by natriuretic peptides, and the soluble guanylate cyclases which can be stimulated by NO. The soluble guanylate cyclases consist of two subunits and very probably contain one haem per heterodimer, which is part of the regulatory site. The latter is of central importance for the mechanism of activation. NO is able to bind to the iron atom of haem and thus markedly increase the activity of the enzyme. Haem-free preparations cannot, by contrast, be stimulated by NO. Carbon monoxide (CO) is also able to attach to the central iron atom of haem, but the stimulation by CO is distinctly less than that by NO.

Through the production of cGMP and the regulation, resulting therefrom, of phosphodiesterases, ion channels and protein kinases, guanylate cyclase plays a crucial part in various physiological processes, in particular in the relaxation and proliferation of smooth muscle cells, in platelet aggregation and adhesion and in neuronal signal transmission, and in disorders caused by an impairment of the aforementioned processes. Under pathophysiological conditions, the NO/cGMP system may be suppressed, which may lead for example to high blood pressure, platelet activation, increased cellular proliferation, endothelial dysfunction, atherosclerosis, angina pectoris, heart failure, myocardial infarction, thromboses, stroke and sexual dysfunction.

A possible way of treating such disorders which is independent of NO and aims at influencing the cGMP signaling pathway in organisms is a promising approach because of the high efficiency and few side effects which are to be expected.

Compounds, such as organic nitrates, whose effect is based on NO have to date been exclusively used for the therapeutic stimulation of soluble guanylate cyclase. NO is produced by bioconversion and activates soluble guanylate cyclase by attaching to the central iron atom of haem. Besides the side effects, the development of tolerance is one of the crucial disadvantages of this mode of treatment.

Some substances which directly stimulate soluble guanylate cyclase, i.e. without previous release of NO, have been described in recent years, such as, for example, 3-(5′-hydroxymethyl-2′-furyl)-1-benzylindazole [YC-1, Wu et al., Blood 84 (1994), 4226; Mülsch et al., Brit. J. Pharmacol. 120 (1997), 681], fatty acids [Goldberg et al., J. Biol. Chem. 252 (1977), 1279], diphenyliodonium hexafluorophosphate [Pettibone et al., Eur. J. Pharmacol. 116 (1985), 307], isoliquiritigenin [Yu et al., Brit. J. Pharmacol. 114 (1995), 1587] and various substituted pyrazole derivatives (WO 98/16223).

Fused pyrazole derivatives are described inter alia in WO 98/23619, WO 00/06568, WO 00/06569, WO 02/42299, WO 02/42300, WO 02/42301, WO 02/42302, WO 02/092596, WO 03/004503, WO 03/095451 and WO 2008/031513 as stimulators of soluble guanylate cyclase. However, it has been found that some of these compounds have disadvantages with respect to their physicochemical properties such as, for example, their solubility, or with respect to their in vivo properties, such as, for example, their behaviour in the liver, their pharmacokinetic behaviour, their dose-activity relationship and/or their metabolic path.

Furthermore, WO 03/076408 describes indazole derivatives for the treatment of cardiovaskular disorders and disorders of the central nervous system. WO 03/035005 discloses heteroindanes as cannabinoid mimetics for the treatment of pain and neurodegenerative disorders. WO 2007/075847 claims 3-aminoindazoles for the treatment of metabolic disorders.

It was an object of the present invention to provide novel substances which act as stimulators of soluble guanylate cyclase and, compared to the compounds known from the prior art, have the same or an improved physicochemical, pharmacokinetic and/or therapeutic profile.

The present invention provides compounds of the general formula (I)

L-A-M-Q (I)

in which
A represents O, S, —S(═O)—, S(═O)₂— or NR¹,
   where
   R¹ represents hydrogen or (C₁-C₄)-alkyl,
L represents (C₅-C₇)-cycloalkyl, phenyl, pyridyl, pyrimidinyl, furyl, thienyl, thiazolyl, oxazolyl, isothiazolyl or isoxazolyl,
   where phenyl, pyridyl, pyrimidinyl, furyl, thienyl, thiazolyl, oxazolyl, isothiazolyl and isoxazolyl may be substituted by 1 or 2 substituents independently of one another selected from the group consisting of halogen, cyano, (C₁-C₄)-alkyl, trifluoromethyl, chloromethyl and (C₂-C₄)-alkynyl
   and
   where (C₅-C₇)-cycloalkyl may be substituted by 1 or 2 substituents independently of one another selected from the group consisting of fluorine and (C₁-C₄)-alkyl,
M represents a bicyclic heteroaryl group of the formula

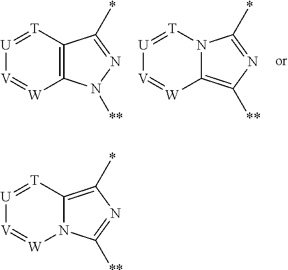

where
* represents the point of attachment to group A,
** represents the point of attachment to group Q,
T, U, V and W each represent CR² or N,
   with the proviso that at most two of the ring members T, U, V and W simultaneously represent N,
   and
   in which
   R² represents hydrogen, halogen, cyano, (C₁-C₄)-alkyl, trifluoromethyl, amino, (C₁-C₄)-alkoxy or trifluoromethoxy,
   and
   in which, if the substituent R² occurs more than once, its meanings may be identical or different
and
Q represents an unsaturated 5- or 6-membered heterocycle or a 5- or 6-membered heteroaryl,
   where the 5- or 6-membered heterocycle and the 5- or 6-membered heteroaryl may be substituted by 1 to 4 substituents independently of one another selected from the group consisting of halogen, azido, nitro, cyano, oxo, thioxo, —R³, —C(═O)—R³, —C(═O)—OR³, —C(═O)—NR³R⁴, —O—(C═O)ₙ—R³, —O—C(═O)—OR³, —O—C(═O)—NR³R⁴, —S(O)ₚ—R³, —SO₂—OR³, —SO₂—NR³R⁴, —NR³—(C═O)ₙ—R⁴, —NR³—SO₂—R⁴, —NR³—C(═O)—OR⁴, —NR⁵—C(═O)—NR³R⁴ and —NR⁵—SO₂—NR³R⁴
      in which
      n represents a number 0 or 1,
      p represents a number 0, 1 or 2,
      R³, R⁴ and R⁵ each independently of one another represent hydrogen, (C₁-C₆)-alkyl, (C₂-C₆)-alkenyl, (C₃-C₈)-cycloalkyl, (C₃-C₈)-cycloalkenyl, (C₆-C₁₀)-aryl, 4- to 8-membered heterocyclyl or 5- to 10-membered heteroaryl,
         in which R³, R⁴ and R⁵ for their part may be substituted by 1 to 5 substituents independently of one another selected from the group consisting of halogen, azido, nitro, cyano, trifluoromethyl, (C₁-C₆)-alkyl, (C₁-C₆)-alkylcarbonyl, (C₁-C₆)-alkylcarbonyloxy, hydroxycarbonyl, (C₁-C₆)-alkoxycarbonyl, aminocarbonyl, mono-(C₁-C₆)-alkylaminocarbonyl, di-(C₁-C₆)-alkylaminocarbonyl, hydroxyl, trifluoromethoxy, (C₁-C₆)-alkoxy, oxo, mercapto, (C₁-C₆)-alkylthio, amino, mono-(C₁-C₆)-alkylamino, di-(C₁-C₆)-alkylamino, formylamino, (C₁-C₆)-alkylcarbonylamino, (C₁-C₆)-alkoxycarbonylamino, (C₃-C₈)-cycloalkyl, (C₃-C₈)-cycloalkenyl and 4- to 8-membered heterocyclyl,
      or
      R³ and R⁴ together with the radical to which the two are attached form a 4- to 8-membered heterocycle,
      or
      R³ and R⁵ together with the radical to which the two are attached form a 4- to 8-membered heterocycle,
         and their N-oxides, salts, solvates, salts of the N-oxides and solvates of the N-oxides and salts.

Compounds according to the invention are the compounds of the formula (I) and the salts, solvates and solvates of the salts thereof, the compounds which are encompassed by formula (I) and are of the formulae mentioned hereinafter, and the salts, solvates and solvates of the salts thereof, and the compounds which are encompassed by formula (I) and are mentioned hereinafter as exemplary embodiments, and the salts, solvates and solvates of the salts thereof, insofar as the compounds encompassed by formula (I) and mentioned hereinafter are not already salts, solvates and solvates of the salts.

Compounds according to the invention also include N-oxides of the compounds of the formula (I) and their salts, solvates and solvates of the salts.

The compounds according to the invention may, depending on their structure, exist in stereoisomeric forms (enantiomers, diastereomers). The present invention therefore relates to the enantiomers or diastereomers and respective mixtures thereof The stereoisomerically pure constituents can be isolated in a known manner from such mixtures of enantiomers and/or diastereomers.

Where the compounds according to the invention can occur in tautomeric forms, the present invention encompasses all tautomeric forms.

Salts preferred for the purposes of the present invention are physiologically acceptable salts of the compounds according to the invention. However, salts which are themselves unsuitable for pharmaceutical applications but can be used for example for isolating or purifying the compounds according to the invention are also encompassed.

Physiologically acceptable salts of the compounds according to the invention include acid addition salts of mineral acids, carboxylic acids and sulphonic acids, e.g. salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of conventional bases such as, for example and preferably, alkali metal salts (e.g. sodium and potassium salts), alkaline earth metal salts (e.g. calcium and magnesium salts) and ammonium salts derived from ammonia or organic amines having 1 to 16 C atoms, such as, for example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methyl-morpholine, arginine, lysine, ethylenediamine and N-methylpiperidine.

Solvates refer for the purposes of the invention to those forms of the compounds according to the invention which form a complex in the solid or liquid state through coordination with solvent molecules. Hydrates are a specific form of solvates in which the coordination takes place with water. Solvates preferred in the context of the present invention are hydrates.

The present invention also encompasses prodrugs of the compounds according to the invention. The term “prodrugs” encompasses compounds which themselves may be biologically active or inactive but are converted during their residence time in the body into compounds according to the invention (for example by metabolism or hydrolysis).

In the context of the present invention, the substituents have the following meaning unless otherwise specified:

In the context of the invention, alkyl represents a straight-chain or branched alkyl radical having the respective stated number of carbon atoms. The following may be mentioned by way of example and by way of preference: methyl, ethyl, n-propyl, isopropyl, n-butyl, isobutyl, 1-methylpropyl, tert-butyl, n-pentyl, isopentyl, 1-ethylpropyl, 1-methylbutyl, 2-methylbutyl, 3-methylbutyl, n-hexyl, 1-methylpentyl, 2-methylpentyl, 3-methylpentyl, 4-methylpentyl, 3,3-dimethylbutyl, 1-ethylbutyl and 2-ethylbutyl.

In the context of the invention, cycloalkyl represents a monocyclic saturated alkyl radical having 3 to 8 carbon atoms. The following may be mentioned by way of example and by way of preference: cyclopropyl, cyclobutyl, cyclopentyl, cyclohexyl, cycloheptyl and cyclooctyl.

In the context of the invention, alkenyl represents a straight-chain or branched alkenyl radical having 2 to 6 carbon atoms and one or two double bonds. Preference is given to a straight-chain or branched alkenyl radical having 2 to 4 carbon atoms and one double bond. The following may be mentioned by way of example and by way of preference: vinyl, allyl, isopropenyl and n-but-2-en-1-yl.

In the context of the invention, cycloalkenyl represents a monocyclic carbocycle having 3 to 8 carbon atoms and one double bond. The following may be mentioned by way of example and by way of preference: cyclobutenyl, cyclopentenyl, cyclohexenyl, cycloheptenyl and cyclooctenyl.

In the context of the invention, alkynyl represents a straight-chain or branched alkynyl radical having 2 to 4 carbon atoms and a triple bond. The following may be mentioned by way of example and by way of preference: ethynyl, n-prop-1-yn-1-yl, n-prop-2-yn-1-yl, n-but-2-yn-1-yl and n-but-3-yn-1-yl.

In the context of the invention, alkylcarbonyl represents a straight-chain or branched alkyl radical having 1 to 6 or 1 to 4 carbon atoms in the alkyl chain and a carbonyl group attached in the 1-position. The following may be mentioned by way of example and by way of preference: methylcarbonyl, ethylcarbonyl, n-propylcarbonyl, isopropylcarbonyl, n-butylcarbonyl, isobutylcarbonyl and tert-butylcarbonyl.

In the context of the invention, alkylcarbonyloxy represents a straight-chain or branched alkyl-carbonyl radical which is attached via an oxygen atom and which carries 1 to 6 or 1 to 4 carbon atoms in the alkyl chain. The following may be mentioned by way of example and by way of preference: methylcarbonyloxy, ethylcarbonyloxy, n-propylcarbonyloxy, isopropylcarbonyloxy, n-butylcarbonyloxy, isobutylcarbonyloxy and tert-butylcarbonyloxy.

In the context of the invention, alkoxy represents a straight-chain or branched alkoxy radical having 1 to 4 carbon atoms. The following may be mentioned by way of example and by way of preference: methoxy, ethoxy, n-propoxy, isopropoxy, 1-methylpropoxy, n-butoxy, isobutoxy and tert-butoxy.

In the context of the invention, alkylthio represents a straight-chain or branched alkylthio radical having 1 to 6 or 1 to 4 carbon atoms. The following may be mentioned by way of example and by way of preference: methylthio, ethylthio, n-propylthio, isopropylthio, n-butylthio, tert-butylthio, n-pentylthio and n-hexylthio.

In the context of the invention, alkoxycarbonyl represents a straight-chain or branched alkoxy radical having 1 to 6 or 1 to 4 carbon atoms and a carbonyl group attached at the oxygen. Preference is given to a straight-chain or branched alkoxycarbonyl radical having 1 to 4 carbon atoms in the alkoxy group. The following may be mentioned by way of example and by way of preference: methoxycarbonyl, ethoxycarbonyl, n-propoxycarbonyl, isopropoxycarbonyl and tert-butoxycarbonyl.

In the context of the invention, monoalkylamino represents an amino group having a straight-chain or branched alkyl substituent which has 1 to 6 carbon atoms. The following may be mentioned by way of example and by way of preference: methylamino, ethylamino, n-propylamino, isopropylamino and tert-butylamino.

In the context of the invention, dialkylamino represents an amino group having two identical or different straight-chain or branched alkyl substituents having 1 to 6 carbon atoms each. The following may be mentioned by way of example and by way of preference: N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino.

In the context of the invention, monoalkylaminocarbonyl represents an amino group which is attached via a carbonyl group and has a straight-chain or branched alkyl substituent having 1 to 6 or 1 to 4 carbon atoms. Preference is given to a monoalkylaminocarbonyl radical having 1 to 4 carbon atoms in the alkyl group. The following may be mentioned by way of example and by way of preference: methylaminocarbonyl, ethylaminocarbonyl, n-propylaminocarbonyl, isopropyl-aminocarbonyl, n-butylaminocarbonyl, tert-butylaminocarbonyl, n-pentylaminocarbonyl and n-hexylaminocarbonyl.

In the context of the invention, dialkylaminocarbonyl represents an amino group which is attached via a carbonyl group and has two identical or different straight-chain or branched alkyl substituents having 1 to 6 or 1 to 4 carbon atoms each. Preference is given to a dialkylaminocarbonyl radical having 1 to 4 carbon atoms each per alkyl group. The following may be mentioned by way of example and by way of preference: N,N-dimethylaminocarbonyl, N,N-diethylaminocarbonyl, N-ethyl-N-methylaminocarbonyl, N-methyl-N-n-propylaminocarbonyl, N-n-butyl-N-methyl-aminocarbonyl, N-tert-butyl-N-methylaminocarbonyl, N-n-pentyl-N-methylaminocarbonyl and N-n-hexyl-N-methylaminocarbonyl.

In the context of the invention, alkylcarbonylamino represents an amino group having a straight-chain or branched alkylcarbonyl substituent which has 1 to 6 or 1 to 4 carbon atoms in the alkyl chain and is attached via the carbonyl group to the nitrogen atom. The following may be mentioned by way of example and by way of preference: methylcarbonylamino, ethylcarbonylamino, propylcarbonylamino, n-butylcarbonylamino, isobutylcarbonylamino and tert-butylcarbonylamino.

In the context of the invention, alkoxycarbonylamino represents an amino group having a straight-chain or branched alkoxycarbonyl substituent which has 1 to 6 or 1 to 4 carbon atoms in the alkyl chain and is attached via the carbonyl group to the nitrogen atom. The following may be mentioned by way of example and by way of preference: methoxycarbonylamino, ethoxycarbonylamino, propoxycarbonylamino, n-butoxycarbonylamino, isobutoxycarbonylamino and tert-butoxycarbonylamino.

In the context of the invention, aryl represents an aromatic carbocycle having 6 or 10 ring carbon atoms. Preferred aryl radicals are phenyl and naphthyl.

In the context of the invention, 5- to 10-membered heteroaryl represents a mono- or optionally bicyclic aromatic heterocycle (hetero aromatic) having a total of 5 to 10 ring atoms which contains up to three identical or different ring heteroatoms from the group consisting of N, O and S and which is attached via a ring carbon atom or optionally via a ring nitrogen atom. The following may be mentioned by way of example: furyl, pyrrolyl, thienyl, pyrazolyl, imidazolyl, thiazolyl, oxazolyl, isoxazolyl, isothiazolyl, triazolyl, oxadiazolyl, thiadiazolyl, pyridyl, pyrimidinyl, pyridazinyl, pyrazinyl, triazinyl, benzofuranyl, benzothienyl, benzimidazolyl, benzoxazolyl, benzothiazolyl, benzotriazolyl, indolyl, indazolyl, quinolinyl, isoquinolinyl, naphthyridinyl, quinazolinyl, quinoxalinyl, phthalazinyl, pyrazolo[3,4-b]pyridinyl. Preference is given to mono-cyclic 5- or 6-membered heteroaryl radicals having up to three ring heteroatoms from the group consisting of N, O and S such as, for example, furyl, thienyl, thiazolyl, oxazolyl, isothiazolyl, isoxazolyl, pyrazolyl, imidazolyl, triazolyl, oxadiazolyl, thiadiazolyl, pyridyl, pyrimidinyl, pyridazinyl, pyrazinyl, triazinyl.

In the context of the invention, a 4- to 8-membered heterocycle represents a monocyclic saturated heterocycle having a total of 4 to 8 ring atoms which contains one or two ring heteroatoms from the group consisting of N, O, S, SO and SO₂ and which is attached via a ring carbon atom or optionally via a ring nitrogen atom. Preference is given to a 5- to 7-membered heterocycle having one or two ring heteroatoms from the group consisting of N, O and S, particularly preferably a 5- or 6-membered heterocycle having one or two ring heteroatoms from the group consisting of N and O. The following may be mentioned by way of example: azetidinyl, oxetanyl, pyrrolidinyl, pyrazolidinyl, tetrahydrofuranyl, thiolanyl, piperidinyl, piperazinyl, tetrahydropyranyl, tetrahydrothiopyranyl, morpholinyl, thiomorpholinyl, hexahydroazepinyl and hexahydro-1,4-diazepinyl. Preference is given to pyrrolidinyl, tetrahydrofuranyl, piperidinyl, piperazinyl, tetrahydropyranyl and morpholinyl.

In the context of the invention, an unsaturated 5- or 6-membered heterocycle represents a monocyclic heterocycle which has a total of 5 or 6 ring atoms, which contains up to four ring heteroatoms from the group consisting of N, O and S, which is attached via a ring carbon atom or, if appropriate, a ring nitrogen atom and, in the case of the five-membered ring, contains one double bond and, in the case of the six-membered ring, contains one or two double bonds. The following may be mentioned by way of example: pyrrolinyl, dihydropyrazolyl, imidazolinyl, dihydrooxazolyl, dihydroisoxazolyl, dihydro-1,2,4-triazolyl, dihydro-1,2,4-oxadiazolyl, dihydro-1,3,4-oxadiazolyl, dihydro-1,2,4-thiadiazolyl, dihydropyranyl, 1,4-dihydropyridyl, tetrahydro-pyrimidinyl, 1,3-oxazinyl.

In the context of the invention, halogen includes fluorine, chlorine, bromine and iodine. Preference is given to chlorine or fluorine.

In the context of the invention, an oxo group represents an oxygen atom which is attached via a double bond to a carbon atom.

If radicals in the compounds according to the invention are substituted, the radicals may, unless otherwise specified, be substituted one or more times. In the context of the present invention, all radicals which occur more than once have a mutually independent meaning. Substitution by one, two or three identical or different substituents is preferred.

Preference is given in the context of the present invention to compounds of the formula (I) in which
A represents O, S or NR¹,
   where
   R¹ represents hydrogen,
L represents phenyl, thienyl, pyridyl or pyrimidinyl,
   where phenyl, thienyl, pyridyl and pyrimidinyl may be substituted by 1 or 2 substituents independently of one another selected from the group consisting of fluorine, chlorine, cyano, methyl, ethyl and trifluoromethyl,
M represents a bicyclic heteroaryl group of the formula

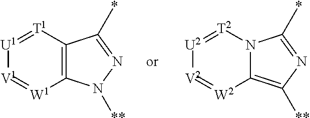

in which
* represents the point of attachment to group A,
** represents the point of attachment to group Q,
T¹, U¹, V¹ and W¹ each represent CR^{2A} or N,
   where at most two of the ring members T¹, U¹, V¹ and W¹ simultaneously represent N
   and
   where
   R^{2A} represents hydrogen or fluorine,
   where at most two of the radicals R^{2A} represent fluorine,
   and
   where, if the substituent R^{2A} occurs more than once, its meanings may be identical or different,
T², U², V² and W² each represent CR^{2B} or N,
   where at most two of the ring members T², U², V² and W² simultaneously represent N
   and
   where
   R^{2B} represents hydrogen or fluorine,
   where at most two of the radicals R^{2B} represent fluorine,
   and
   where, if the substituent R^{2B} occurs more than once, its meanings may be identical or different,
and
Q represents a group of the formula

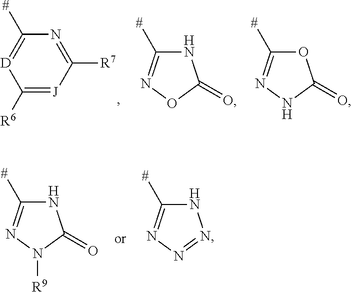

where
# represents the point of attachment to group M,
D represents CH or N,
J represents CR⁸, N or N⁺—O⁻,
   in which
   R⁸ represents halogen, nitro, cyano, —R³, —C(═O)—R³, —C(═O)—OR³, —C(═O)—NR³R⁴, —O—(C═O)ₙ—R³, —O—C(═O)—OR³, —O—C(═O)—NR³R⁴, —S(O)ₚ—R³, —SO₂—OR³, —SO₂—NR³R⁴, ‘3NR³—(C═O)ₙ—R⁴, —NR³—SO₂—R⁴, —NR³—C(═O)—OR⁴, —NR⁵—C(═O)—NR³R⁴ or —NR⁵—SO₂—NR³R⁴,
      in which
      n represents a number 0 or 1,
      p represents a number 0 or 2,
      R³, R⁴ and R⁵ each independently of one another represent hydrogen, (C₁-C₆)-alkyl, (C₂-C₆)-alkenyl, (C₃-C₇)-cycloalkyl, (C₃-C₇)-cycloalkenyl, phenyl, 5- to 7-membered heterocyclyl or 5- or 6-membered heteroaryl,
         in which R³, R⁴ and R⁵ for their part may be substituted by 1 to 3 substituents independently of one another selected from the group consisting of fluorine, chlorine, cyano, (C₁-C₄)-alkyl, trifluoromethyl, hydroxyl, (C₁-C₄)-alkoxy, trifluoromethoxy, oxo, amino, mono-(C₁-C₄)-alkylamino and di-(C₁-C₄)-alkylamino,
      or
      R³ and R⁴ together with the radical to which the two are attached may form a 5- to 7-membered heterocycle,
      or
      R³ and R⁵ together with the radical to which the two are attached may form a 5- to 7-membered heterocycle,
R⁹ represents hydrogen, (C₁-C₆)-alkyl or (C₃-C₇)-cycloalkyl,
   where (C₁-C₆)-alkyl may be substituted by 1 to 5 substituents independently of one another selected from the group consisting of (C₃-C₇)-cycloalkyl, hydroxyl, (C₁-C₄)-alkoxy, trifluoromethoxy, (C₁-C₄)-acyloxy, amino, mono-(C₁-C₄)-alkylamino, di-(C₁-C₄)-alkylamino, (C₁-C₄)-acylamino, hydroxycarbonyl, (C₁-C₄)-alkoxycarbonyl, aminocarbonyl, mono-(C₁-C₄)-aminocarbonyl, di-(C₁-C₄)-alkylaminocarbonyl and a 5- or 6-membered heterocycle,
      and their N-oxides, salts, solvates, salts of the N-oxides and solvates of the N-oxides and salts.

Particular preference is given in the context of the present invention to compounds of the formula (I) in which
A represents S or NR¹,
   where
   R¹ represents hydrogen,
L represents phenyl, pyridyl or pyrimidinyl,
   where phenyl may be substituted by 1 or 2 fluorine substituents,
M represents a bicyclic heteroaryl group of the formula

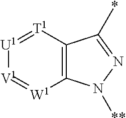

in which
* represents the point of attachment to group A,
** represents the point of attachment to group Q,
and
T¹ represents CH or N,
U¹ represents CH,
W¹ represents CH,
V¹ represents CR^{2A}
   in which
   R^{2A} represents hydrogen or fluorine,
Q represents a group of the formula

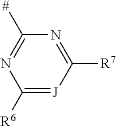

where
# represents the point of attachment to group M,
J represents CR⁸ or N,
   in which
   R⁸ represents hydrogen, fluorine, (C₁-C₄)-alkyl, (C₃-C₇)-cycloalkyl, phenyl, pyridyl, —NR³—(C═O)ₙ—R⁴, —NR³—C(═O)—OR⁴ or —NR⁵—C(═O)—NR³R⁴
      in which
      n represents the number 0 or 1,
      R³ represents hydrogen or (C₁-C₄)-alkyl,
         in which (C₁-C₄)-alkyl for its part may be substituted by a substituent selected from the group consisting of fluorine, trifluoromethyl, hydroxyl and methoxy,
      R⁴ represents hydrogen, (C₁-C₄)-alkyl or (C₃-C₇)-cycloalkyl, in which (C₁-C₄)-alkyl for its part may be substituted by a substituent selected from the group consisting of fluorine, trifluoromethyl, hydroxyl and methoxy,
      R⁵ represents hydrogen or (C₁-C₄)-alkyl,
      R³ and R⁴ together with the radical to which the two are attached may form a 5- to 7-membered heterocycle,
R⁶ represents hydrogen or amino,
R⁷ represents hydrogen or amino,
   and their N-oxides, salts, solvates, salts of the N-oxides and solvates of the N-oxides and salts.

Preference is also given to compounds of the formula (I) in which
Q represents a group of the formula

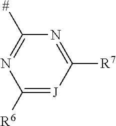

where
# represents the point of attachment to group M,
J represents CR⁸ or N,
   in which
   R⁸ represents pyridyl or —NR³—C(═O)—OR⁴,
      in which
      R³ represents hydrogen or (C₁-C₄)-alkyl,
      R⁴ represents hydrogen or (C₁-C₄)-alkyl,
         in which (C₁-C₄)-alkyl for its part may be substituted by a substituent selected from the group consisting of fluorine, trifluoromethyl, hydroxyl and methoxy,
R⁶ represents hydrogen or amino,
R⁷ represents hydrogen or amino

Preference is also given to compounds of the formula (I) in which
L represents phenyl,
   where phenyl may be substituted by one fluorine substituent.

Preference is also given to compounds of the formula (I) in which
M represents a bicyclic heteroaryl group of the formula

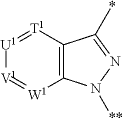

in which
* represents the point of attachment to group A,
** represents the point of attachment to group Q, and
T¹ represents CH or N,
U¹ represents CH,
W¹ represents CH,
V¹ represents CR^{2A}
   in which
   R^{2A} represents hydrogen or fluorine.

Preference is also given to compounds of the formula (I) in which
M represents a bicyclic heteroaryl group of the formula

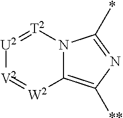

in which
* represents the point of attachment to group A,
** represents the point of attachment to group Q,
and
T² represents CH or N,
U² represents CH,
W² represents CH,
V² represents CR^{2B} or N
   in which
   R^{2B} represents hydrogen or fluorine.

The definitions of radicals indicated specifically in the respective combinations or preferred combinations of radicals are replaced as desired irrespective of the particular combinations indicated for the radicals also by definitions of radicals of other combinations.

Combinations of two or more of the abovementioned preferred ranges are particularly preferred.

Analogously to methods described in the literature, the compounds of the formula (I) according to the invention can be prepared, for example, by
[A] reacting a compound of the formula (II)

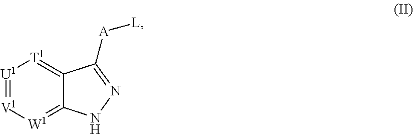

in which A, L, T¹, U¹, V¹ and W¹ each have the meanings given above, in an inert solvent in the presence of a palladium catalyst and a suitable base with a compound of the formula (III)

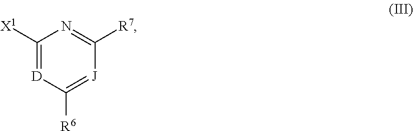

in which D, J, R⁶ and R⁷ each have the meanings given above,
and
X¹ represents a suitable leaving group such as, for example, halogen, mesylate, tosylate or triflate
to give a compound of the formula (I-A)

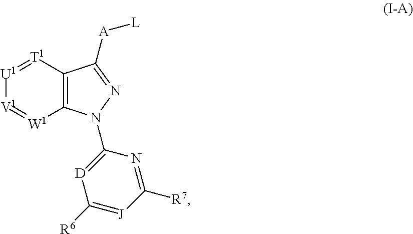

in which A, D, J, L, T¹, U¹, V¹, W¹, R⁶ and R⁷ each have the meanings given above, or
[B] converting a compound of the formula (IV)

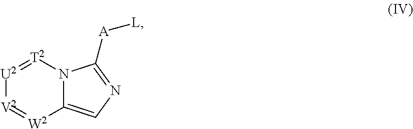

in which A, L, T², U², V² and W² each have the meanings given above, in an inert solvent with a halogenating agent into a compound of the formula (V)

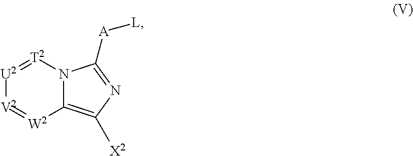

in which A, L, T², U², V² and W² each have the meanings given above
and
X² represents halogen, in particular bromine,
and then by standard methods into a tin species (VI)

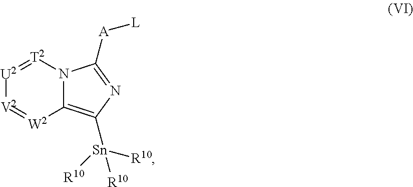

in which A, L, T², U², V² and W² each have the meanings given above
and
R¹⁰ represents (C₁-C₄)-alkyl,
which is then reacted in an inert solvent in the presence of a palladium catalyst and a suitable base with a compound of the formula (III)
to give a compound of the formula (I-B)

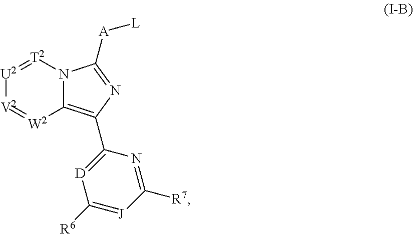

in which A, D, J, L, T², U², V², W², R⁶ and R⁷ each have the meanings given above,
or
[C] reacting a compound of the formula (VII)

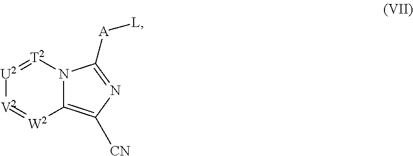

in which A, L, T², U², V² and W² each have the meanings given above,
in an inert solvent in the presence of a suitable base with a compound of the formula (VIII)

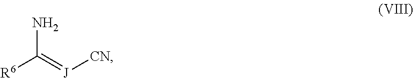

in which J and R⁶ each have the meanings given above,
to give a compound of the formula (I-C)

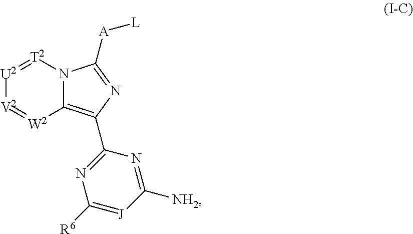

in which A, J, L, T², U², V², W² and R⁶ each have the meanings given above,
   modifying, if appropriate, the resulting compounds of the formulae (I-A), (I-B) and (I-C) according to processes known from the literature further within the above scope of the meanings of the individual substituents and radicals and/or converting the compounds according to the invention obtained in this manner, if appropriate, with the appropriate (i) solvents and/or (ii) acids or bases into their solvates, salts and/or solvates of the salts.

Inert solvents for process steps (II)+(III)→(I-A) and (VI)+(III)→(I-B) are, for example, alcohols, such as methanol, ethanol, n-propanol, isopropanol, n-butanol or tert-butanol, ethers, such as diethyl ether, dioxane, tetrahydrofuran, glycol dimethyl ether or diethylene glycol dimethyl ether, hydrocarbons, such as benzene, xylene, toluene, hexane, cyclohexane or mineral oil fractions, or other solvents, such as dimethylformamide (DMF), dimethyl sulphoxide (DMSO), N,N′-dimethyl-propyleneurea (DMPU), N-methylpyrrolidone (NMP), pyridine, acetonitrile or else water. It is also possible to use mixtures of the solvents mentioned. Dimethylformamide and toluene and a mixture of dimethylformamide and toluene are preferred.

Bases suitable for process steps (II)+(III)→(I-A) and (VI)+(III)→(I-B) are customary inorganic bases. These include in particular alkali metal hydroxides, such as, for example, lithium hydroxide, sodium hydroxide or potassium hydroxide, alkali metal bicarbonates, such as sodium bicarbonate or potassium bicarbonate, alkali metal or alkaline earth metal carbonates, such as lithium carbonate, sodium carbonate, potassium carbonate, calcium carbonate or caesium-carbonate, or alkali metal hydrogen phosphates, such as disodium hydrogen phosphate or dipotassium hydrogen phosphate. Preference is given to using caesium carbonate.

Suitable palladium catalysts for process steps (II)+(III)→(I-A) and (VI)+(III)→(I-B) are, for example, palladium on activated carbon, palladium(II) acetate, tetrakis(triphenylphosphine)-palladium(0), bis(triphenylphosphine)palladium(II) chloride, bis(acetonitrile)palladium(II) chloride and [1,1′-bis(diphenylphosphino)ferrocene]dichloropalladium(II)/dichloromethane complex, if appropriate in combinations with additional phosphane ligands such as, for example, (2-biphenyl)di-tert.-butylphosphine, dicyclohexyl[2′,4′,6′-tris(1-methylethyl)biphenyl-2-yl]phosphane (XPHOS), bis(2-phenylphosphinophenyl)ether (DPEphos) or 4,5-bis(diphenylphosphino)-9,9-dimethylxanthene (Xantphos) [cf., for example, Hassan J. et al., Chem. Rev. 102, 1359-1469 (2002)].

The reactions (II)+(III)→(I-A) and (VI)+(III)→(I-B) are generally carried out in a temperature range of from +20° C. to +180° C., preferably at from +50° C. to +120° C., if appropriate in a microwave. The reaction can be carried out at atmospheric, elevated or reduced pressure (for example in the range of from 0.5 to 5 bar). In general, the reaction is carried out at atmospheric pressure.

Inert solvents for process step (VII)+(VIII)→(I-C) are, for example, alcohols, such as methanol, ethanol, n-propanol, isopropanol, n-butanol and tert-butanol, ethers, such as diethyl ether, dioxane, tetrahydrofuran, glycol dimethyl ether or diethylene glycol dimethyl ether, hydrocarbons, such as benzene, xylene, toluene, hexane, cyclohexane or mineral oil fractions, or other solvents, such as dimethylformamide (DMF), dimethyl sulphoxide (DMSO), N,N′-dimethylpropyleneurea (DMPU), N-methylpyrrolidone (NMP), pyridine, acetonitrile or else water. It is also possible to use mixtures of the solvents mentioned. Dimethylformamide is preferred.

Bases suitable for these reactions are the customary inorganic or organic bases. These preferably include alkali metal hydrides, such as sodium hydride, alkali metal hydroxides, such as, for example, lithium hydroxide, sodium hydroxide or potassium hydroxide, alkali metal carbonates, such as lithium carbonate, sodium carbonate, potassium carbonate or caesium carbonate, alkali metal bicarbonates, such as sodium bicarbonate or potassium bicarbonate, alkali metal alkoxides, such as sodium methoxide or potassium methoxide, sodium ethoxide or potassium ethoxide or potassium tert-butoxide, amides, such as sodium amide, lithium bis(trimethylsilyl)amide, sodium bis(trimethylsilyl)amide or potassium bis(trimethylsilyl)amide or lithium diisopropylamide, organometallic compounds, such as butyllithium or phenyllithium, or organic amines, such as triethylamine, diisopropylethylamine, pyridine, 1,8-diazabicyclo[5.4.0]undec-7-ene (DBU) or 1,5-diazabicyclo[4.3.0]non-5-ene (DBN). Preference is given to sodium methoxide and triethylamine.

The reaction (VII)+(VIII)→(I-C) is generally carried out in a temperature range of from +20° C. to +180° C., preferably from +50° C. to +120° C., if appropriate in a microwave. The reaction can be carried out at atmospheric, elevated or reduced pressure (for example in the range of from 0.5 to 5 bar). In general, the reaction is carried out at atmospheric pressure.

The compounds of the formula (III) are commercially available, known from the literature or can be prepared analogously to processes known from the literature.

Starting with compounds of the formula (IV) or (VII), compounds of the formula (I-B) or (I-C) can also be prepared analogously to the processes mentioned in WO 03/095451 and WO 2008/031513.

A compound of the formula (II) in which A represents S can be prepared by reacting a compound of the formula (IX)

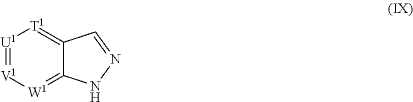

in which T¹, U¹, V¹ and W¹ each have the meanings given above
in an inert solvent with a suitable halogenating agent, in particular with bromine, to give a compound of the formula (X)

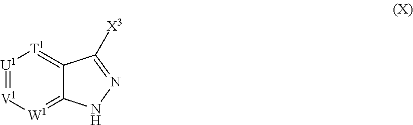

in which T¹, U¹, V¹ and W¹ each have the meanings given above,
and
X³ represents halogen, in particular bromine or iodine,
and then reacting this in an inert solvent in the presence of a suitable catalyst and a suitable base with a compound of the formula (XI)

HS-L (XI),
in which L has the meaning given above,
to give a compound (II-A)

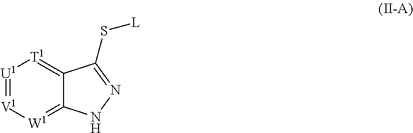

in which L, T¹, U¹, V¹ and W¹ each have the meanings given above.

The preparation process described can be illustrated in an exemplary manner by the synthesis scheme below (Scheme 1):

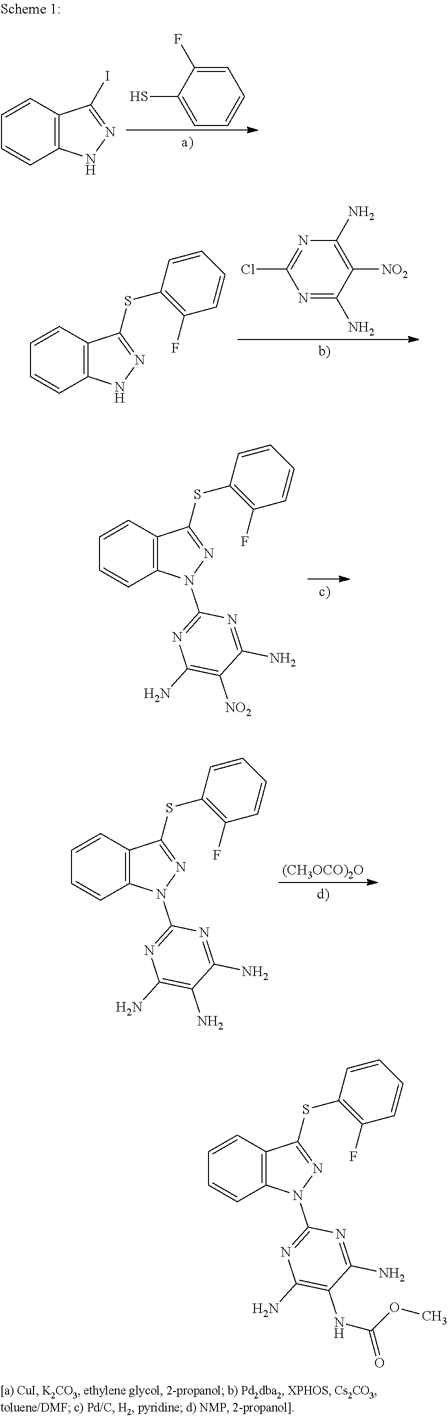

The compounds of the formulae (IX) and (XI) are commercially available, known from the literature or can be prepared analogously to processes known from the literature.

A compound of the formula (II) in which A represents NH can be prepared by reacting a compound of the formula (XII)

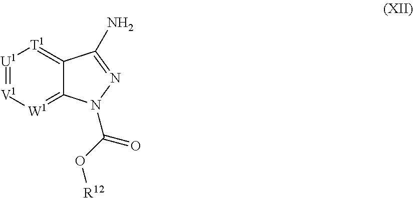

in which T¹, U¹, V¹ and W¹ each have the meanings given above
and
R¹² represents (C₁-C₄)-alkyl,
in an inert solvent in the presence of a suitable catalyst and a suitable base with a compound of the formula (XIII)

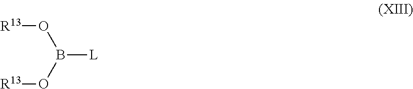

in which L has the meaning given above
and
R¹³ represents hydrogen or both radicals R¹³ together form a —C(CH₃)₂—C(CH₃)₂— or —CH2—C(CH₃)₂—CH₂— bridge,
to give a compound of the formula (XIV)

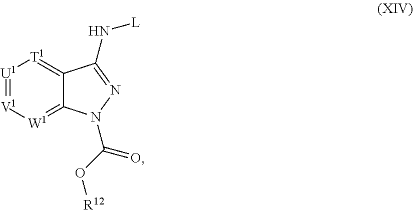

in which L, T¹, U¹, V¹, W¹ and R¹² each have the meanings given above,
which, after removal of the carbamate under standard conditions, gives a compound (II-B)

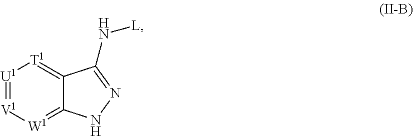

in which L, T¹, U¹, V¹ and W¹ each have the meanings given above.

The compounds of the formulae (XII) and (XIII) are commercially available, known from the literature or can be prepared analogously to processes known from the literature.

The preparation process described can be illustrated in an exemplary manner by the synthesis scheme below (Scheme 2):

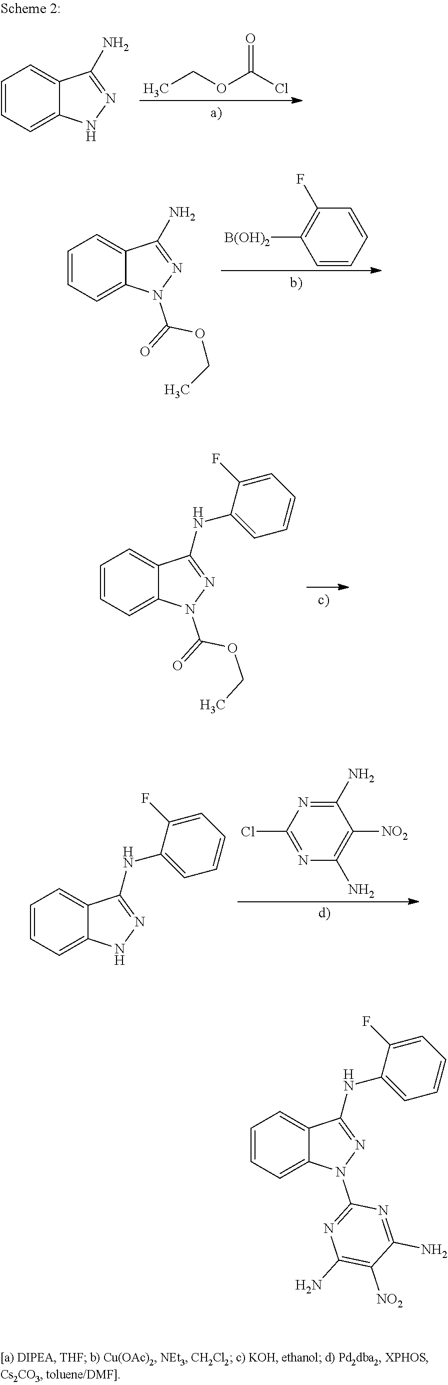

Compounds of the formula (II) in which A represents 0 can be prepared analogously to the process described in Examples 12A to 16A and as illustrated in an exemplary manner in the synthesis scheme below (Scheme 3).

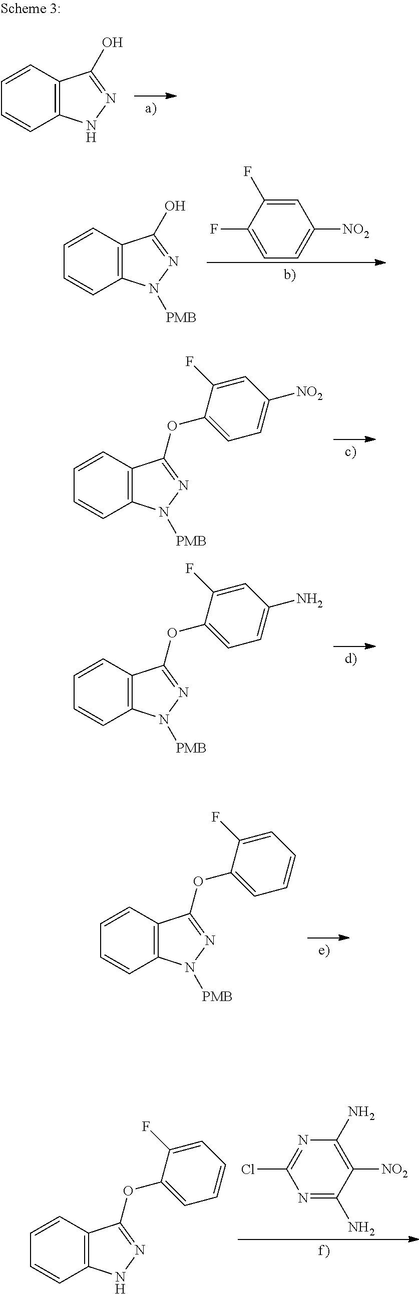

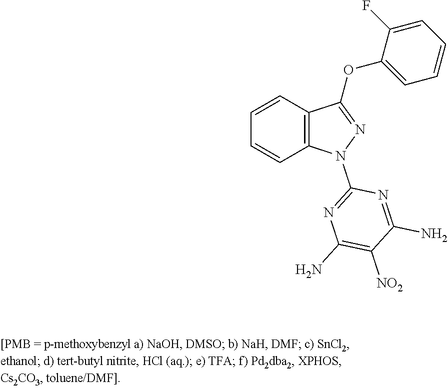 [SMILES: Nc1nc(-n2nc(Oc3ccccc3F)c3ccccc32)nc(N)c1[N+](=O)[O-]]

Compounds of the formula (I) in which M represents a group of the formula

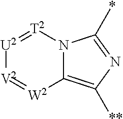

where *, **, T², U², V² and W² each have the meanings given above can be prepared analogously to processes known from the literature [cf., for example, Bourdais J. et al., J. Heterocyclic Chem. 1980, 17, 555; Bourdais J. et al., J. Heterocyclic Chem. 1980, 17, 1351; WO 2004/074290; WO 2005/080391], or as shown in an exemplary manner in the synthesis schemes below:

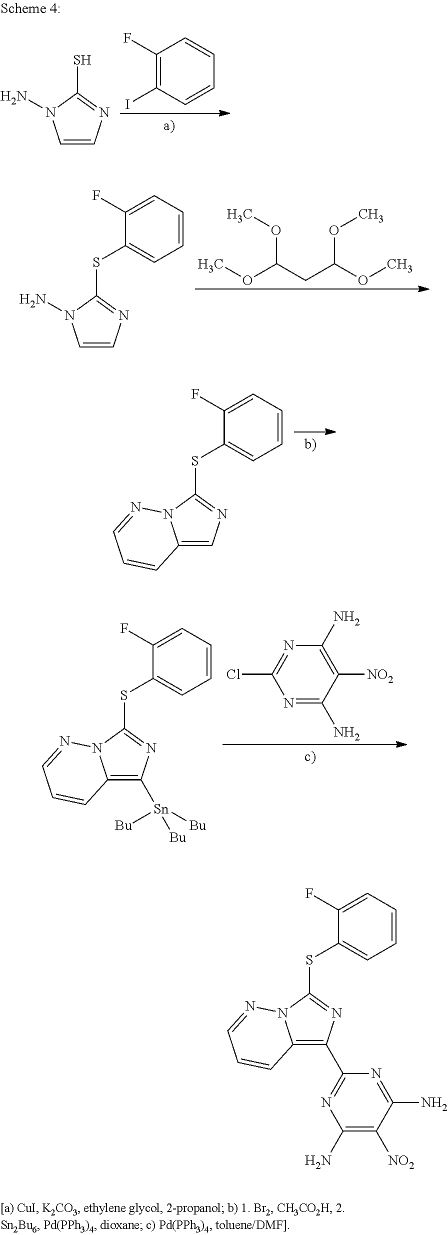

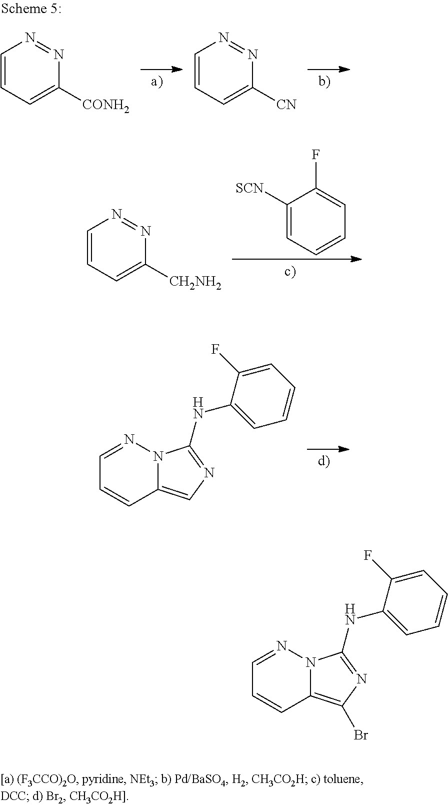

Compounds of the formula (I) in which Q represents a group of the formula

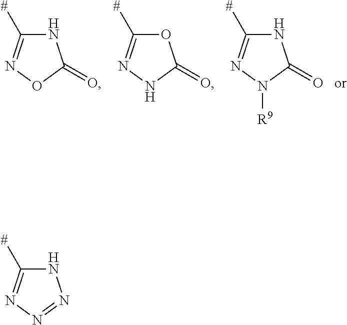

where R⁹ has the meaning given above can be prepared analogously to the processes described in WO 2008/031513.

The compounds according to the invention have valuable pharmacological properties and can be used for the prevention and treatment of disorders in humans and animals. The compounds according to the invention offer a further treatment alternative and therefore enlarge pharmacy.

The compounds according to the invention lead to vasorelaxation, to an inhibition of platelet aggregation and to a reduction in blood pressure, and also to an increase in coronary blood flow. These effects are mediated by direct stimulation of soluble guanylate cyclase and an increase in intracellular cGMP. Moreover, the compounds according to the invention enhance the effect of substances increasing the cGMP concentration, such as, for example, EDRF (endothelium-derived relaxing factor), NO donors, protoporphyrin IX, arachidonic acid or phenylhydrazine derivatives.

The compounds according to the invention can therefore be employed in medicaments for the treatment of cardiovascular disorders such as, for example, for the treatment of high blood pressure and heart failure, stable and unstable angina pectoris, pulmonary hypertension, peripheral and cardiac vascular disorders, arrhythmias, for the treatment of thromboembolic disorders and ischemias such as myocardial infarction, stroke, transistoric and ischemic attacks, disturbances of peripheral blood flow, reperfusion damage, prevention of restenoses as after thrombolysis therapies, percutaneous transluminal angioplasties (PTAs), percutaneous transluminal coronary angioplasties (PTCAs), bypass and for the treatment of arteriosclerosis, asthmatic disorders, erectile dysfunction, female sexual dysfunction, osteoporosis, glaucoma, and gastroparesis.

The compounds according to the invention can additionally be used for the treatment of primary and secondary Raynaud's phenomenon, of microcirculation impairments, claudication, peripheral and autonomic neuropathies, diabetic microangiopathies, diabetic retinopathy, diabetic ulcers on the extremities, gangrenes, CREST syndrome, erythematosis, onychomycosis, rheumatic disorders and for promoting wound healing.

The compounds according to the invention are furthermore suitable for treating urological disorders such as, for example, benign prostate syndrome (BPS), benign prostate hyperplasia (BPH), benign prostate enlargement (BPE), bladder outlet obstruction (BOO), lower urinary tract syndromes (LUTS), disorders of the urogenital system including neurogenic over-active bladder (OAB) and (IC), incontinence (UI) such as, for example, mixed urinary incontinence, urge urinary incontinence, stress urinary incontinence or overflow urinary incontinence (MUI, UUI, SUI, OUI), pelvic pain, benign and malignant disorders of the organs of the male and femal urogenital system, kidney disorders such as, for example, acute or chronic renal failure, immunological kidney disorders such as kidney transplant rejection, glumerulonephritis, immune complex-induced kidney disorders, glomerulopathies, nephritis, toxic nephropathy and obstructive uropathies.

The compounds according to the invention are furthermore suitable for the treatment of acute and chronic lung diseases, such as respiratory distress syndromes (ALI, ARDS) and chronic obstructive airway disorders (COPD), and also for the treatment of acute and chronic renal failure.

The compounds described in the present invention also represent active ingredients for controlling central nervous system diseases characterized by disturbances of the NO/cGMP system. They are suitable in particular for improving perception, concentration, learning or memory after cognitive impairments like those occurring in particular in association with situations/diseases/syndromes such as mild cognitive impairment, age-associated learning and memory impairments, age-associated memory losses, vascular dementia, craniocerebral trauma, stroke, dementia occuring after strokes (post stroke dementia), post-traumatic craniocerebral trauma, general concentration impairments, concentration impairments in children with learning and memory problems, Alzheimer's disease, Lewy body dementia, dementia with degeneration of the frontal lobes including Pick's syndrome, Parkinson's disease, progressive nuclear palsy, dementia with corticobasal degeneration, amyolateral sclerosis (ALS), Huntington's disease, multiple sclerosis, thalamic degeneration, Creutzfeld-Jacob dementia, HIV dementia, schizophrenia with dementia or Korsakoff s psychosis. They are also suitable for the treatment of central nervous system disorders such as states of anxiety, tension and depression, CNS-related sexual dysfunctions and sleep disturbances, and for controlling pathological disturbances of the intake of food, stimulants and addictive substances.

The compounds according to the invention are furthermore also suitable for controlling cerebral blood flow and thus represent effective agents for controlling migraine. They are also suitable for the prophylaxis and control of the sequelae of cerebral infarctions (Apoplexia cerebri) such as stroke, cerebral ischemias and craniocerebral trauma. The compounds according to the invention can likewise be employed for controlling states of pain and tinnitus.

In addition, the compounds according to the invention have an anti-inflammatory effect and can therefore be employed as anti-inflammatory agents.

The present invention further relates to the use of the compounds according to the invention for the treatment and/or prevention of disorders, especially of the aforementioned disorders.

The present invention further relates to the compounds according to the invention for use in a method for the treatment and/or prevention of heart failure, angina pectoris, hypertension, pulmonary hypertension, ischemias, vascular disorders, thromboembolic disorders and arteriosclerosis.

The present invention further relates to the use of the compounds according to the invention for producing a medicament for the treatment and/or prevention of disorders, especially of the aforementioned disorders.

The present invention further relates to a method for the treatment and/or prevention of disorders, especially of the aforementioned disorders, by using an effective amount of at least one of the compounds according to the invention.

The compounds according to the invention can be employed alone or, if required, in combination with other active ingredients. The present invention further relates to medicaments comprising at least one of the compounds according to the invention and one or more further active ingredients, in particular for the treatment and/or prevention of the aforementioned disorders. Active ingredients suitable for combinations are, by way of example and by way of preference:
organic nitrates and NO donors such as, for example, sodium nitroprusside, nitroglycerin, isosorbide mononitrate, isosorbide dinitrate, molsidomine or SIN-1, and inhaled NO;
compounds which inhibit the breakdown of cyclic guanosine monophosphate (cGMP), such as, for example, inhibitors of phosphodiesterases (PDE) 1, 2 and/or 5, in particular PDE 5 inhibitors such as sildenafil, vardenafil and tadalafil;
agents having antithrombotic activity, for example and preferably from the group of platelet aggregation inhibitors, of anticoagulants or of profibrinolytic substances;
active ingredients which lower blood pressure, for example and preferably from the group of calcium antagonists, angiotensin All antagonists, ACE inhibitors, endothelin antagonists, renin inhibitors, alpha-receptor blockers, beta-receptor blockers, mineralocorticoid receptor antagonists, and of diuretics; and/or
active ingredients which modify lipid metabolism, for example and preferably from the group of thyroid receptor agonists, cholesterol synthesis inhibitors such as, for example and preferably, HMG-CoA reductase inhibitors or squalene synthesis inhibitors, of ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein (a) antagonists.

Agents having antithrombotic activity preferably mean compounds from the group of platelet aggregation inhibitors, of anticoagulants or of profibrinolytic substances.

In a preferred embodiment of the invention, the compounds according to the invention are administered in combination with a platelet aggregation inhibitor such as, for example and preferably, aspirin, clopidogrel, ticlopidin or dipyridamole.

In a preferred embodiment of the invention, the compounds according to the invention are administered in combination with a thrombin inhibitor such as, for example and preferably, ximelagatran, melagatran, bivalirudin or clexane.

In a preferred embodiment of the invention, the compounds according to the invention are administered in combination with a GPIIb/IIIa antagonist such as, for example and preferably, tirofiban or abciximab.

In a preferred embodiment of the invention, the compounds according to the invention are administered in combination with a factor Xa inhibitor such as, for example and preferably, rivaroxaban (BAY 59-7939), DU-176b, apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds according to the invention are administered in combination with heparin or with a low molecular weight (LMW) heparin derivative.

In a preferred embodiment of the invention, the compounds according to the invention are administered in combination with a vitamin K antagonist such as, for example and preferably, coumarin.

Agents which lower blood pressure preferably mean compounds from the group of calcium antagonists, angiotensin All antagonists, ACE inhibitors, endothelin antagonists, renin inhibitors, alpha-receptor blockers, beta-receptor blockers, mineralocorticoid receptor antagonists, and of diuretics.

In a preferred embodiment of the invention, the compounds according to the invention are administered in combination with a calcium antagonist such as, for example and preferably, nifedipine, amlodipine, verapamil or diltiazem.

In a preferred embodiment of the invention, the compounds according to the invention are administered in combination with an alpha-1-receptor blocker such as, for example and preferably, prazosin.

In a preferred embodiment of the invention, the compounds according to the invention are administered in combination with a beta-receptor blocker such as, for example and preferably, propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds according to the invention are administered in combination with an angiotensin All antagonist such as, for example and preferably, losartan, candesartan, valsartan, telmisartan or embursatan.

In a preferred embodiment of the invention, the compounds according to the invention are administered in combination with an ACE inhibitor such as, for example and preferably, enalapril, captopril, lisinopril, ramipril, delapril, fosinopril, quinopril, perindopril or trandopril.

In a preferred embodiment of the invention, the compounds according to the invention are administered in combination with an endothelin antagonist such as, for example and preferably, bosentan, darusentan, ambrisentan or sitaxsentan.

In a preferred embodiment of the invention, the compounds according to the invention are administered in combination with a renin inhibitor such as, for example and preferably, aliskiren, SPP-600 or SPP-800.

In a preferred embodiment of the invention, the compounds according to the invention are administered in combination with a mineralocorticoid receptor antagonist such as, for example and preferably, spironolactone or eplerenone.

In a preferred embodiment of the invention, the compounds according to the invention are administered in combination with a diuretic such as, for example and preferably, furosemide.

Agents which modify lipid metabolism preferably mean compounds from the group of CETP inhibitors, thyroid receptor agonists, cholesterol synthesis inhibitors such as HMG-CoA reductase inhibitors or squalene synthesis inhibitors, of ACAT inhibitors, MTP inhibitors, PPAR-alpha, PPAR-gamma and/or PPAR-delta agonists, cholesterol absorption inhibitors, polymeric bile acid adsorbents, bile acid reabsorption inhibitors, lipase inhibitors and of lipoprotein(a) antagonists.

In a preferred embodiment of the invention, the compounds according to the invention are administered in combination with a CETP inhibitor such as, for example and preferably, dalcetrapib, BAY 60-5521, anacetrapib or CETP vaccine (CETi-1).

In a preferred embodiment of the invention, the compounds according to the invention are administered in combination with a thyroid receptor agonist such as, for example and preferably, D-thyroxine, 3,5,3′-triiodothyronine (T3), CGS 23425 or axitirome (CGS 26214).

In a preferred embodiment of the invention, the compounds according to the invention are administered in combination with an HMG-CoA reductase inhibitor from the class of statins such as, for example and preferably, lovastatin, simvastatin, pravastatin, fluvastatin, atorvastatin, rosuvastatin or pitavastatin.

In a preferred embodiment of the invention, the compounds according to the invention are administered in combination with a squalene synthesis inhibitor such as, for example and preferably, BMS-188494 or TAK-475.

In a preferred embodiment of the invention, the compounds according to the invention are administered in combination with an ACAT inhibitor such as, for example and preferably, avasimibe, melinamide, pactimibe, eflucimibe or SMP-797.

In a preferred embodiment of the invention, the compounds according to the invention are administered in combination with an MTP inhibitor such as, for example and preferably, implitapide, BMS-201038, R-103757 or JTT-130.

In a preferred embodiment of the invention, the compounds according to the invention are administered in combination with a PPAR-gamma agonist such as, for example and preferably, pioglitazone or rosiglitazone.

In a preferred embodiment of the invention, the compounds according to the invention are administered in combination with a PPAR-delta agonist such as, for example and preferably, GW 501516 or BAY 68-5042.

In a preferred embodiment of the invention, the compounds according to the invention are administered in combination with a cholesterol absorption inhibitor such as, for example and preferably, ezetimibe, tiqueside or pamaqueside.

In a preferred embodiment of the invention, the compounds according to the invention are administered in combination with a lipase inhibitor such as, for example and preferably, orlistat.

In a preferred embodiment of the invention, the compounds according to the invention are administered in combination with a polymeric bile acid adsorbent such as, for example and preferably, cholestyramine, colestipol, colesolvam, CholestaGel or colestimide.

In a preferred embodiment of the invention, the compounds according to the invention are administered in combination with a bile acid reabsorption inhibitor such as, for example and preferably, ASBT (=IBAT) inhibitors such as, for example, AZD-7806, S-8921, AK-105, BARI-1741, SC-435 or SC-635.

In a preferred embodiment of the invention, the compounds according to the invention are administered in combination with a lipoprotein (a) antagonist such as, for example and preferably, gemcabene calcium (CI-1027) or nicotinic acid.

The present invention further relates to medicaments which comprise at least one compound according to the invention, normally together with one or more inert, non-toxic, pharmaceutically suitable excipients, and to the use thereof for the aforementioned purposes.

The compounds according to the invention can act systemically and/or locally. For this purpose, they can be administered in a suitable way such as, for example, by the oral, parenteral, pulmonal, nasal, sublingual, lingual, buccal, rectal, dermal, transdermal, conjunctival, otic route or as implant or stent.

The compounds according to the invention can be administered in administration forms suitable for these administration routes.

Suitable for oral administration are administration forms which function according to the prior art and deliver the compounds according to the invention rapidly and/or in modified fashion, and which contain the compounds according to the invention in crystalline and/or amorphized and/or dissolved form, such as, for example, tablets (uncoated or coated tablets, for example having enteric coatings or coatings which are insoluble or dissolve with a delay and control the release of the compound according to the invention), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration are, inter alia, preparations for injection and infusion in the form of solutions, suspensions, emulsions, lyophilizates or sterile powders.

Suitable for the other administration routes are, for example, pharmaceutical forms for inhalation (inter alia powder inhalers, nebulizers), nasal drops, solutions or sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (e.g. patches), milk, pastes, foams, dusting powders, implants or stents.

Oral or parenteral administration is preferred, especially oral administration.

The compounds according to the invention can be converted into the stated administration forms. This can take place in a manner known per se by mixing with inert, non-toxic, pharmaceutically suitable excipients. These excipients include, inter alia, carriers (for example microcrystalline cellulose, lactose, mannitol), solvents (e.g. liquid polyethylene glycols), emulsifiers and dispersants or wetting agents (for example sodium dodecyl sulphate, polyoxysorbitan oleate), binders (for example polyvinylpyrrolidone), synthetic and natural polymers (for example albumin), stabilizers (e.g. antioxidants such as, for example, ascorbic acid), colorants (e.g. inorganic pigments such as, for example, iron oxides) and masking flavours and/or odours.

It has generally proved advantageous to administer on parenteral administration amounts of about 0.001 to 1 mg/kg, preferably about 0.01 to 0.5 mg/kg, of body weight to achieve effective results, and on oral administration the dosage is about 0.01 to 100 mg/kg, preferably about 0.01 to 20 mg/kg, and very particularly preferably 0.1 to 10 mg/kg, of body weight.

It may nevertheless be necessary where appropriate to deviate from the stated amounts, in particular as a function of the body weight, route of administration, individual response to the active ingredient, nature of the preparation and time or interval over which administration takes place. Thus, it may be sufficient in some cases to make do with less than the aforementioned minimum amount, whereas in other cases the stated upper limit must be exceeded. It may in the event of administration of larger amounts be advisable to divide these into a plurality of individual doses over the day.

The following exemplary embodiments illustrate the invention. The invention is not restricted to the examples.

The percentage data in the following tests and examples are, unless indicated otherwise, percentages by weight; parts are parts by weight. Solvent ratios, dilution ratios and concentration data for the liquid/liquid solutions are in each case based on volume.

### A. EXAMPLES

Abbreviations and Acronyms:

aq. aqueous solution

ber. calculated

DCI direct chemical ionization (in MS)

DMAP 4-N,N-dimethylaminopyridine

DMF dimethylformamide

DMSO dimethyl sulphoxide

eq. equivalent(s)

ESI electrospray ionization (in MS)

Et ethyl

h hour(s)

HPLC high-pressure, high-performance liquid chromatography

HRMS high-resolution mass spectrometry

conc. concentrated

LC/MS liquid chromatography-coupled mass spectroscopy

LiHMDS lithium hexamethyldisilazide

Me methyl

min minute(s)

MS mass spectrometry

NMR nuclear magnetic resonance spectroscopy

Pd₂dba₃ tris(dibenzylideneacetone)dipalladium

Ph phenyl

RT room temperature

Rₜ retention time (in HPLC)

THF tetrahydrofuran

UV ultraviolet spectrometry

v/v ratio by volume (of a solution)

XPHOS dicyclohexyl-(2′,4′,6′-triisopropylbiphenyl-2-yl)phosphine

LC/MS and HPLC Methods:

Method 1 (Preparative HPLC):

Gilson Abimed HPLC; binary pump system; column: ReproSil C18, 250×30; mobile phase A: water/0.5% ammonia, mobile phase B: acetonitrile; gradient: 0-3 min 60% B, 3.01-35 min 95% B, 35-40 min 95% B; flow rate: 50 ml/min; UV detection at 210 nm.

Method 2 (LC-MS):

Instrument: Micromass QuattroPremier with Waters UPLC Acquity; column: Thermo Hypersil GOLD 1.9μ 50 mm×1 mm; mobile phase A: 1 l of water+0.5 ml of 50% strength formic acid, mobile phase B: 1 l of acetonitrile+0.5 ml of 50% strength formic acid; gradient: 0 0 min 90% A→0.1 min 90% A→1.5 min 10% A→2.2 min 10% A; flow rate: 0.33 ml/min; oven: 50° C.; UV detection: 210 nm.

Method 3 (LC-MS):

Instrument: Micromass Quattro Micro MS with HPLC Agilent Series 1100; column: Thermo Hypersil GOLD 3μ, 20 mm×4 mm; mobile phase A: 1 l of water +0.5 ml of 50% strength formic acid, mobile phase B: 1 l of acetonitrile+0.5 ml of 50% strength formic acid; gradient 0.0 min 100% A→3.0 min 10% A→4.0 min 10% A→4.01 min 100% A (flow rate 2.5 ml/min)→5.00 min 100% A; oven: 50° C.; flow rate: 2 ml/min; UV detection: 210 nm.

Method 4 (LC-MS):

MS instrument type: Micromass ZQ; HPLC instrument type: Waters Alliance 2795; column: Phenomenex Synergi 2.5μ MAX-RP 100A Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→0.1 min 90% A→3.0 min 5% A→4.0 min 5% A→4.01 min 90% A; flow rate: 2 ml/min; oven: 50° C.; UV detection: 210 nm.

Method 5 (LC-MS):

MS instrument type: Micromass ZQ; HPLC instrument type: HP 1100 Series; UV DAD; column: Phenomenex Gemini 3μ 30 mm×3.00 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

Method 6 (LC-MS):

Instrument: Waters ACQUITY SQD UPLC System; column: Waters Acquity UPLC HSS T3 1.8μ 50×1 mm; mobile phase A: 1 l of water+0.25 ml of 99% strength formic acid, mobile phase B: 1 l of acetonitrile+0.25 ml of 99% strength formic acid; gradient: 0.0 min 90% A→1.2 min 5% A→2.0 min 5% A; oven: 50° C.; flow rate: 0.40 ml/min; UV detection: 210-400 nm.

Starting Materials and Intermediates:

### Example 1A

### 3-[(2-Fluorophenyl)sulphanyl]-1H-pyrazolo[4,3-b]pyridine

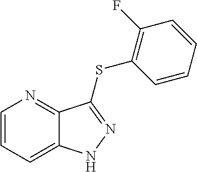 [SMILES: Fc1ccccc1Sc1n[nH]c2cccnc12]

Under an atmosphere of argon, 4.84 ml (86.77 mmol) of 1,2-ethanediol, 250 ml of 2-propanol and 11.12 g (86.77 mmol) of 2-fluorothiophenol were added to 10.63 g (43.38 mmol) of 3-iodo-1H-pyrazolo[4,3-b]pyridine, 1.24 g (6.51 mmol) of copper(I) iodide and 12.0 g (86.77 mmol) of potassium carbonate, and the mixture was stirred at 130° C. for 20 hours. The salts were filtered off with suction, silica gel was added to the filtrate, the mixture was concentrated and the residue was chromatographed on a silica gel column using a gradient of cyclohexane/ethyl acetate=10:1 to 1:1. This gave 11.55 g (98% of theory) of the product in a purity of 90% (HPLC).

LC-MS (Method 6): Rₜ=0.83 min

MS (ESIpos): m/z=246.1 (M+H)⁺

¹H-NMR (400 MHz, DMSO-d₆): δ=6.9-7.1 (m, 2H), 7.2-7.3 (m, 2H), 7.46 (dd, 1H), 8.10 (d, 1H), 8.55 (d, 1H), 13.88 (broad s, 1H).

### Example 2A

### 2-{3-[(2-Fluorophenyl)sulphanyl]-1H-pyrazolo[4,3-b]pyridin-1-yl}-5-nitropyrimidine-4,6-diamine

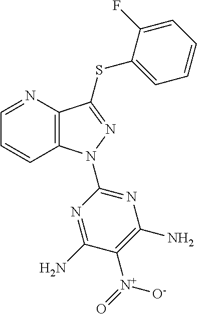 [SMILES: Nc1nc(-n2nc(Sc3ccccc3F)c3ncccc32)nc(N)c1[N+](=O)[O-]]

Under argon, 3 g (12.23 mmol) of 3-[(2-fluorophenyl)sulphanyl]-1H-indazole, 3.48 g (18.35 mmol) of 2-chloro-5-nitropyrimidine-4,6-diamine [Bitterli et al., Helv. Chim. Acta 1951, 34, 835], 560 mg (0.612 mmol) of tris(dibenzylideneacetone)dipalladium, 583 mg (1.22 mmol) of dicyclohexyl(2′,4′,6′-triisopropylbiphenyl-2-yl)phosphane and 5.58 g (17.12 mmol) of caesium carbonat were stirred in 50 ml of toluene and 50 ml of DMF at 90° C. for 18 hours. The mixture was allowed to cool to room temperature, the solid was filtered off with suction and washed with THF, the filtrate was concentrated under reduced pressure and the evaporation residue was triturated with a THF/water mixture. The product was then concentrated under reduced pressure and suspended in dichloromethane/methanol, silica gel was added and the mixture was concentrated under reduced pressure and chromatographed on a silica gel column using a gradient of dichloromethane/ethanol=100:1 to 50:1. This gave, in a purity of 45% (HPLC), 2.73 g (25.2% of theory) of the target compound which was used directly for the next step (Example 1).

LC-MS (Method 2): Rₜ=1.05 min

MS (ESIpos): m/z=399.0 (M+H)⁺

¹H-NMR (400 MHz, DMSO-d₆): δ=7.12 (t, 1H), 7.25-7.4 (m, 3H), 7.6 (dd, 1H), 8.63 (m, 1H), 8.80 (broad s, 2H), 8.98 (broad s, 2H), 9.40 (d, 1H).

### Example 3A

### Ethyl 3-[(2-fluorophenyl)amino]-1H-indazole-l-carboxylate

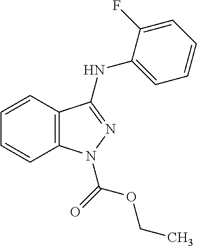 [SMILES: CCOC(=O)n1nc(Nc2ccccc2F)c2ccccc21]

9.77 g (47.61 mmol) of ethyl 3-amino-1H-indazole-l-carboxylate (preparation: DE 2458965, page 28), 9.99 g (71.41 mmol) of 2-fluorophenylboronic acid, 0.95 g (4.76 mmol) of copper(II) acetate monohydrate and 7.532 g (95.22 mmol) of pyridine were stirred in 329 ml of DMF at 20° C. for 18 hours. 1.8 1 of water were then added, and the mixture was extracted five times with ethyl acetate. The combined organic extracts were washed three times with saturated aqueous sodium chloride solution, dried with sodium sulphate and concentrated under reduced pressure. The residue was purified on silica gel using a cyclohexane/ethyl acetate gradient and the product-containing fraction was concentrated. This gave 1.34 g (9.2% of theory) of the target compound.

LC-MS (Method 2): Rₜ=1.34 min

MS (ESIpos): m/z=300.3 (M+H)⁺

¹H-NMR (400 MHz, CDCl₃): δ=1.39 (t, 3H), 4.45 (q, 2H), 7.0-7.1 (m, 1H), 7.21 (dd, 1H), 7.29 (d, 1H), 7.38 (t, 1H), 7.62 (t, 1H), 8.09 (d, 1H), 8.20-8.32 (m, 2H), 9.01 (s, 1H).

### Example 4A

### N-(2-Fluorophenyl)-1H-indazole-3-amine

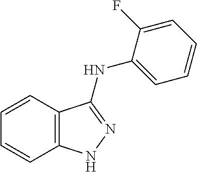 [SMILES: Fc1ccccc1Nc1n[nH]c2ccccc12]

1.1 g (19.7 mmol) of potassium hydroxide were added to 1.47 g (4.92 mmol) of ethyl 3-[(2-fluorophenyl)amino]-1H-indazole-l-carboxylate in 14.7 ml of ethanol, and the mixture was heated at reflux for 10 minutes. 1.182 g (19.69 mmol) of acetic acid were then added, and the mixture was concentrated under reduced pressure. The residue was purified on silica gel using a cyclohexane/ethyl acetate gradient (1:0→1:1) and the product-containing fraction was concentrated. This gave 0.98 g (80% of theory) of the target compound in a purity of 91% (HPLC).

LC-MS (Method 4): Rₜ=1.81 min

MS (ESIpos): m/z=228.4 (M+H)⁺

¹H-NMR (400 MHz, CDCl₃): δ=6.82 (m, 1H), 7.01 (dd, 1H), 7.08 (dd, 1H), 7.18 (dd, 1H), 7.32 (dd, 1H), 7.39 (d, 1H), 7.98 (d, 1H), 8.03 (dd, 1H), 8.35 (s, 1H), 12.12 (s, 1H).

### Example 5A

### 2-{3-[(2-Fluorophenyl)amino]-1H-indazol-1-yl}-5-nitropyrimidine-4,6-diamine

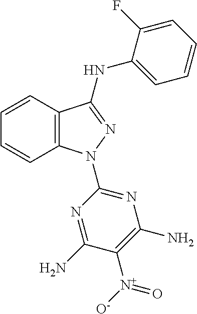 [SMILES: Nc1nc(-n2nc(Nc3ccccc3F)c3ccccc32)nc(N)c1[N+](=O)[O-]]

Under argon, 980 mg (4.31 mmol) of N-(2-fluorophenyl)-1H-indazole-3-amine, 817 mg (4.31 mmol) of 2-chloro-5-nitropyrimidine-4,6-diamine [Bitterli et al., Helv. Chim. Acta 1951, 34, 835], 79 mg (0.086 mmol) of tris(dibenzylideneaceton)dipalladium, 103 mg (0.216 mmol) of dicyclohexyl(2′,4′,6′-triisopropylbiphenyl-2-yl)phosphane and 1.97 g (6.04 mmol) of caesium carbonate were stirred in 16.3 ml of toluene and 16.3 ml of DMF at 90° C. for four hours. The mixture was allowed to cool to room temperature, the solid was filtered off with suction and washed with THF, and the filtrate was concentrated under reduced pressure and chromatographed on a silica gel column using a gradient of cyclohexane/ethyl acetate=5:1 to 0:1. This gave 929 mg (51% of theory) of the target compound in a purity of 89% (HPLC).

LC-MS (Method 5): Rₜ=2.30 min

MS (ESIpos): m/z=381.2 (M+H)⁺

¹H-NMR (400 MHz, DMSO-d₆): δ=7.02 (m, 1H), 7.20 (dd, 1H), 7.25 (dd, 1H), 7.34 (dd, 1H), 7.54 (dd, 1H), 8.20 (d, 1H), 8.40 (dd, 1H), 8.70 (broad d, 4H), 8.91 (s, 1H), 8.99 (d, 1H).

### Example 6A

### 3-[(2-Fluorophenyl)sulphanyl]-1H-indazole

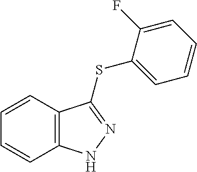 [SMILES: Fc1ccccc1Sc1n[nH]c2ccccc12]

Under an atmosphere of argon, 480 ml of 2-propanol, 10.17 g (164 mmol) of 1,2-ethanediol and 21 g (164 mmol) of 2-fluorothiophenol were added to 20 g (82 mmol) of 3-iodo-1H-indazole, 22.65 g (164 mmol) of potassium carbonate and 2.34 g (12.29 mmol) of copper(I) iodide, and the mixture was stirred at 130° C. for 20 hours. The reaction was cooled to room temperature, silica gel was added and the mixture was concentrated under reduced pressure and chromatographed on silica gel using a cyclohexane/ethyl acetate gradient (10:1→4:1). Evaporation of the product-containing fractions gave 19.9 g (96% of theory) of the target compound in a purity of 97% (HPLC).

LC-MS (Method 2): Rₜ=1.22 min

MS (ESIpos): m/z=245.2 (M+H)⁺

¹H-NMR (400 MHz, DMSO-d₆): δ=6.95 (dd, 1H), 7.02 (m, 1H), 7.18 (dd, 1H), 7.23-7.30 (m, 2H), 7.42 (t, 1H), 7.51 (d, 1H), 7.62 (d, 1H), 13.65 (broad s, 1H).

### Example 7A

### 2-{3-[(2-Fluorophenyl)sulphanyl]-1H-indazol-1-yl}-5-nitropyrimidine-4,6-diamine

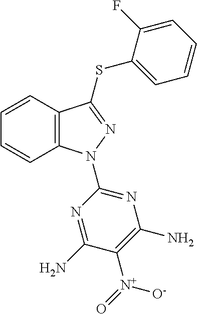 [SMILES: Nc1nc(-n2nc(Sc3ccccc3F)c3ccccc32)nc(N)c1[N+](=O)[O-]]

Under argon, 3 g (12.28 mmol) of 3-[(2-fluorophenyl)sulphanyl]-1H-indazole, 4.66 g (24.6 mmol) of 2-chloro-5-nitropyrimidine-4,6-diamine [Bitterli et al., Helv. Chim. Acta 1951, 34, 835], 225 mg (0.246 mmol) of tris(dibenzylideneacetone)dipalladium, 351 mg (0.74 mmol) of dicyclohexyl(2′,4′,6′-triisopropylbiphenyl-2-yl)phosphane and 8.00 g (24.6 mmol) of caesium carbonate were stirred in 50 ml of toluene and 50 ml of DMF at 90° C. for 17 hours. The mixture was allowed to cool to room temperature and the solid was filtered off with suction. The solid was then triturated with 150 ml of 1N hydrochloric acid. After filtration with suction, the residue was dried under high vacuum. This gave 4.03 g (82.5% of theory) of the target compound.

LC-MS (Method 2): Rₜ=1.32 min

MS (ESIpos): m/z=398.1 (M+H)⁺

¹H-NMR (400 MHz, DMSO-d₆): δ=7.18 (dd, 1H), 7.28-7.42 (m, 4H), 7.44 (d, 1H), 7.59 (dd, 1H), 8.78 (s, 2H), 8.9-9.02 (broad s, 2H), 9.08 (d, 1H).

### Example 8A

### 3-(Pyridin-2-ylsulphanyl)-1H-indazole

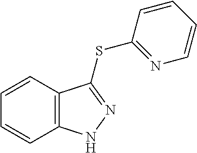 [SMILES: c1ccc(Sc2n[nH]c3ccccc23)nc1]

Under inert conditions, 36 ml of 2-propanol, 763 mg (12.3 mmol) of 1,2-ethanediol and 6.83 g (61.5 mmol) of 2-mercaptopyridine were added to 1.5 g (6.15 mmol) of 3-iodo-1H-indazole, 1.7 g (12.3 mmol) of potassium carbonate and 176 mg (0.922 mmol) of copper(I) iodide, and the mixture was stirred at 130° C. for 44 hours. The reaction was cooled to room temperature, silica gel was added and the mixture was concentrated under reduced pressure and chromatographed on silica gel using a dichloromethane/methanol gradient (50:1→40:1). Evaporation of the product-containing fractions gave 703 mg (47% of theory) of the target compound in a purity of 94% (HPLC).

LC-MS (Method 6): Rₜ=0.86 min

MS (ESIpos): m/z=228.2 (M+H)⁺

¹H-NMR (400 MHz, DMSO-d₆): δ=6.77 (d, 1H), 7.13 (m, 1H), 7.19 (dd, 1H), 7.44 (dd, 1H), 7.50-7.61 (m, 2H), 7.66 (d, 1H), 8.35 (d, 1H), 13.75 (s, 1H).

### Example 9A

### 5-Nitro-2-[3-(pyridin-2-ylsulphanyl)-1H-indazol-1-yl]pyrimidine-4,6-diamine

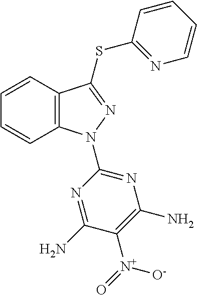 [SMILES: Nc1nc(-n2nc(Sc3ccccn3)c3ccccc32)nc(N)c1[N+](=O)[O-]]

Under argon, 700 mg (3.1 mmol) of 3-(pyridin-2-ylsulphanyl)-1H-indazole, 1.17 g (6.16 mmol) of 2-chloro-5-nitropyrimidine-4,6-diamine [Bitterli et al., Helv. Chim. Acta 1951, 34, 835], 56 mg (0.062 mmol) of tris(dibenzylideneacetone)dipalladium, 88 mg (0.19 mmol) of dicyclohexyl(2′,4′,6′-triisopropylbiphenyl-2-yl)phosphane and 2 g (6.2 mmol) of caesium carbonate were stirred in 12.5 ml of toluene and 12.5 ml of DMF at 90° C. for 17 hours. The mixture was allowed to cool to room temperature and the solid was filtered off with suction. The solid was then triturated with 50 ml of 1N hydrochloric acid. After filtration with suction, the residue was washed with 30 ml of saturated aqueous sodium bicarbonate solution and 50 ml of water and then dried under high vacuum. This gave 668 mg (55% of theory) of the target compound.

LC-MS (Method 6): Rₜ=0.94 min

MS (ESIpos): m/z=381.0 (M+H)⁺

¹H-NMR (400 MHz, DMSO-d₆): δ=7.12 (d, 1H), 7.21 (m, 1H), 7.35 (dd, 1H), 7.51 (d, 1H), 7.57-7.71 (m, 2H), 8.38 (m, 1H), 8.8 (broad s, 2H), 8.97 (broad s, 2H), 9.08 (d, 1H).

### Example 10A

### 3-(Pyrimidin-2-ylsulphanyl)-1H-indazole

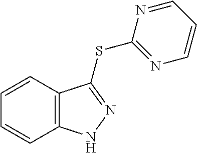 [SMILES: c1cnc(Sc2n[nH]c3ccccc23)nc1]

Under an atmosphere of argon, 36 ml of 2-propanol, 763 mg (12 3 mmol) of 1,2-ethanediol and 6.83 g (61.5 mmol) of 2-mercaptopyrimidine were added to 1.5 g (6.15 mmol) of 3-iodo-1H-indazole, 1.7 g (12.3 mmol) of potassium carbonate and 176 mg (0.922 mmol) of copper(I) iodide, and the mixture was stirred at 130° C. for 44 hours. The reaction was cooled to room temperature, silica gel was added, the mixture was concentrated under reduced pressure and the residue was chromatographed on silica gel using a dichloromethane/methanol mobile phase (20:1). Evaporation of the product-containing fractions gave 554 mg (32% of theory) of the target compound in a purity of 81% (HPLC).

LC-MS (Method 6): Rₜ=0.78 min

MS (ESIpos): m/z=229.2 (M+H)⁺

¹H-NMR (400 MHz, DMSO-d₆): δ=7.18 (dd, 1H), 7.22 (t, 1H), 7.41 (dd, 1H), 7.52 (d, 1H), 7.62 (d, 1H), 8.53 (d, 2H), 13.65 (s, 1H).

### Example 11A

### 5-Nitro-2-[3-(pyrimidin-2-ylsulphanyl)-1H-indazol-1-yl]pyrimidine-4,6-diamine

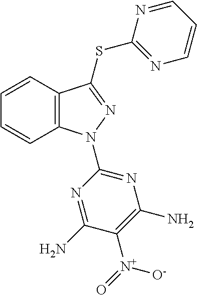 [SMILES: Nc1nc(-n2nc(Sc3ncccn3)c3ccccc32)nc(N)c1[N+](=O)[O-]]

Under argon, 670 mg (2.94 mmol) of 3-(pyrimidin-2-ylsulphanyl)-1H-indazole, 1.67 g (8.8 mmol) of 2-chloro-5-nitropyrimidine-4,6-diamine [Bitterli et al., Helv. Chim. Acta 1951, 34, 835], 108 mg (0.117 mmol) of tris(dibenzylideneacetone)dipalladium, 210 mg (0.44 mmol) of dicyclohexyl(2′,4′,6′-triisopropylbiphenyl-2-yl)phosphane and 1.9 g (5.87 mmol) of caesium carbonate were stirred in 12 ml of toluene and 12 ml of DMF at 100° C. for 17 hours. A further 556 mg of 2-chloro-5-nitropyrimidine-4,6-diamine, 54 mg (0.059 mmol) of tris(dibenzylideneacetone)dipalladium and 126 mg of dicyclohexyl(2′,4′,6′-triisopropylbiphenyl-2-yl)phosphane were then added, and the mixture was stirred at 110° C. for a further 20 h. The mixture was allowed to cool to room temperature and the solid was filtered off with suction. The filtrate was concentrated, taken up in acetonitrile and filtriert. The filter cake was then triturated with 50 ml of 4N hydrochloric acid, filtriert, washed with 30 ml of saturated aqueous sodium bicarbonate solution and then with 50 ml of water and subsequently dried under high vacuum. This gave 996 mg (84% of theory) of the target compound.

LC-MS (Method 6): Rₜ=0.89 min

MS (ESIpos): m/z=382.1 (M+H)⁺

¹H-NMR (400 MHz, DMSO-d₆): δ=7.28 (dd, 1H), 7.38 (dd, 1H), 7.60 (m, 2H), 8.58 (d, 1H), 8.71, 8.79, 8.82, 8.95 (4 broad s, 6H), 9.06 (d, 1H).

### Example 12A

### 3-(2-Fluoro-4-nitrophenoxy)-1-(4-methoxybenzyl)-1H-indazole

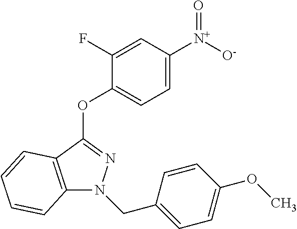 [SMILES: COc1ccc(Cn2nc(Oc3ccc([N+](=O)[O-])cc3F)c3ccccc32)cc1]

0.40 g (1.57 mmol) of 1-(4-methoxybenzyl)-1H-indazol-3-ol (Palazzo, G. et al., J. Med. Chem. 1966, 9(1), 38-41) was dissolved in 4 ml of DMF, 75.5 mg of sodium hydride (60% in mineral oil, 1.89 mmol) were added and the mixture was stirred at RT for 1 h. 0.28 g (1.73 mmol) of 1,2-difluoro-4-nitrobenzene was added to the resulting suspension and the mixture was stirred at RT for a further hour. Water was added, and the mixture was extracted with ethyl acetate. The organic phase was washed with saturated aqueous sodium chloride solution, dried over magnesium sulphate and freed from the solvent on a rotary evaporator. The residue obtained in this manner was purified by preparative HPLC (Method 1). This gave 0.32 g (52% of theory) of the target compound.

LC-MS (Method 2): Rₜ=1.51 min,

MS (ESIpos): m/z=394.1 (M+H)⁺

¹H-NMR (400 MHz, DMSO-d₆): δ=3.70 (s, 3H), 5.50 (s, 2H), 6.87 (d, 2H), 7.17 (t, 1H), 7.21 (d, 2H), 7.43-7.49 (m, 2H), 7.59 (d, 1H), 7.77 (d, 1H), 8.12 (d, 1H), 8.39 (dd, 1H).

### Example 13A

### 3-Fluoro-4-{[1-(4-methoxybenzyl)-1H-indazol-3-yl]oxy}aniline

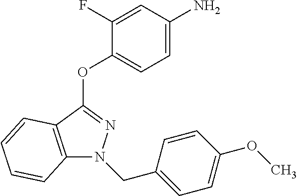 [SMILES: COc1ccc(Cn2nc(Oc3ccc(N)cc3F)c3ccccc32)cc1]

0.31 g (0.79 mmol) of the compound from Example 12A was dissolved in 6 ml of ethanol and 1 ml of DMF, 0.71 g (3.15 mmol) of tin(II) chloride dihydrate was added and the mixture was heated under reflux for 1 h. The mixture was then diluted with 50 ml of 1 N aqueous sodium hydroxide solution and 50 ml of ethyl acetate, and the organic phase was washed with water and saturated aqueous sodium chloride solution, dried over magnesium sulphate and freed from the solvent on a rotary evaporator. The residue obtained in this manner was purified by preparative HPLC (Method 1). This gave 0.18 g (61% of theory) of the target compound.

LC-MS (Method 2): Rₜ=1.29 min

MS (ESIpos): m/z=364.2 (M+H)⁺

¹H-NMR (400 MHz, DMSO-d₆): δ=3.69 (s, 3H), 5.37 (s, 2H), 6.36 (dd, 1H), 6.47 (dd, 1H), 6.84 (d, 2H), 7.00-7.06 (m, 2H), 7.13 (d, 2H), 7.35-7.39 (m, 2H), 7.59 (d, 1H).

### Example 14A

### 3-(2-Fluorophenoxy)-1-(4-methoxybenzyl)-1H-indazole

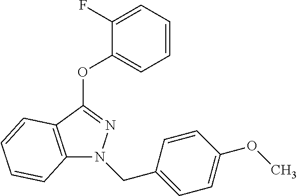 [SMILES: COc1ccc(Cn2nc(Oc3ccccc3F)c3ccccc32)cc1]

89.0 mg (0.86 mmol) of tent-butyl nitrite were initially charged in 1.5 ml of DMF. At 50° C., a solution of the compound from Example 13A in 1 ml of DMF was added dropwise such that the internal temperature did not exceed 50° C. After the addition had ended, the reaction was stirred at 50° C. for a further 30 minutes and then stirred into 6 ml of semiconcentrated hydrochloric acid. The mixture was extracted with ethyl acetate, and the organic phase was washed with saturated aqueous sodium chloride solution, dried over magnesium sulphate and freed from the solvent on a rotary evaporator. The residue obtained in this manner was purified by preparative HPLC (Method 1). This gave 74 mg (49% of theory) of the target compound.

LC-MS (Method 6): Rₜ=1.31 min

MS (ESIpos): m/z=349.3 (M+H)⁺

¹H-NMR (400 MHz, DMSO-d₆): δ=3.69 (s, 3H), 5.42 (s, 2H), 6.85 (d, 2H), 7.10 (t, 1h), 7.16 (d, 2H), 7.19-7.34 (m, 3H), 7.38-7.44 (m, 2H), 7.50 (d, 1H), 7.67 (d, 1H).

### Example 15A

### 3-(2-Fluorophenoxy)-1H-indazole

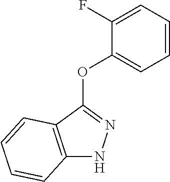 [SMILES: Fc1ccccc1Oc1n[nH]c2ccccc12]

At 70° C., 70 mg (0.2 mmol) of the compound from Example 14A were stirred in 1 ml of trifluoroacetic acid for 1 h. After cooling, the reaction was carefully stirred into 10 ml of saturated aqueous sodium bicarbonate solution and extracted with ethyl acetate, and the organic phase was washed with saturated sodium chloride solution, dried over magnesium sulphate and freed from the solvent on a rotary evaporator. The residue obtained in this manner was purified by preparative HPLC (Method 1). This gave 29 mg (63% of theory) of the target compound.

LC-MS (Method 2): Rₜ=1.17 min,

MS (ESIpos): m/z=229.3 (M+H)⁺

¹H-NMR (400 MHz, DMSO-d₆): δ=7.09 (t, 1h), 7.18-7.32 (m, 3H), 7.40 (m, 2H), 7.47 (d, 1H), 7.50 (d, 1H), 12.39 (s, 1H).

### Example 16A

### 2-[3-(2-Fluorophenoxy)-1H-indazol-1-yl]-5-nitropyrimidine-4,6-diamine

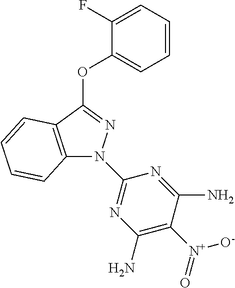 [SMILES: Nc1nc(-n2nc(Oc3ccccc3F)c3ccccc32)nc(N)c1[N+](=O)[O-]]

25 mg (0.11 mmol) of the compound from Example 15A were dissolved in 0.5 ml of DMF, 5.7 mg of sodium hydride (60% in mineral oil, 0.14 mmol) were added and the mixture was stirred at RT for 1 h. A solution of 22.8 mg (0.12 mmol) of 2-chloro-5-nitropyrimidine-4,6-diamine [Bitterli et al., Helv. Chim. Acta 1951, 34, 835] in 1.5 ml of DMF was added to the resulting suspension, and the mixture was stirred at 80° C. for 1 h. The reaction was purified directly by preparative HPLC (Method 1). This gave 23 mg (49% of theory) of the target compound.

LC-MS (Method 6): Rₜ=1.10 min,

MS (ESIpos): m/z=382.3 (M+H)⁺

¹H-NMR (400 MHz, DMSO-d₆): δ=7.28-7.49 (m, 4H), 7.56 (t, 1H), 7.64 (t, 1H), 7.76 (d, 1H), 8.71 (s, 2H), 8.80 (s, 2H), 9.04 (d, 1H).

### WORKING EXAMPLES

### Example 1

### 2-{3-[(2-Fluorophenyl)sulphanyl]-1H-pyrazolo[4,3-b]pyridin-1-yl}pyrimidine-4,5,6-triamine

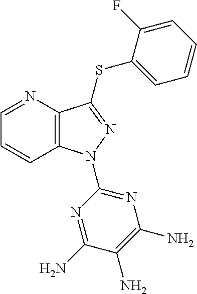 [SMILES: Nc1nc(-n2nc(Sc3ccccc3F)c3ncccc32)nc(N)c1N]

Under a hydrogen pressure of 3.5 bar and at 20° C., 2.727 g (about 3.08 mmol, purity 45%) of 2-{3-[(2-fluorophenyl)sulphanyl]-1H-pyrazolo[4,3-b]pyridin-1-yl}-5-nitropyrimidine-4,6-diamine were shaken with 1.09 g of 10% palladium on activated carbon in 250 ml of pyridine for 17 hours. The reaction was filtered, most of the pyridine was filtered off and the residue was dissolved in acetonitrile. Silica gel was added, and the mixture was concentrated under reduced pressure and chromatographed on a silica gel column using a cyclohexane/ethyl acetate mixture (1:1). This gave 980 mg (31% of theory) of the product in a purity of 79%. A small amount thereof (110 mg) was purified by preparative HPLC (Cromatorex C18 10 μm, 250×30 mm, flow rate 50 ml/min, run time: 38 min, acetonitrile/water gradient+0.1% formic acid). Evaporation of the product-containing fractions gave 76 mg of a solid.

LC-MS (Method 3): Rₜ=1.55 min

MS (ESIpos): m/z=369.2 (M+H)⁺

¹H-NMR (400 MHz, DMSO-d₆): δ=3.88 (s, 2H), 6.15 (s, 4H), 7.03-7.1 (m, 1H), 7.1-7.2 (m, 1H), 7.22-7.35 (m, 2H), 7.53 (dd, 1H), 8.6 (m, 1H), 9.14 (d, 1H).

### Example 2

### Methyl(4,6-diamino-2-{3-[(2-fluorophenyl)amino]-1H-indazol-1-yl}pyrimidin-5-yl)carbamate formate

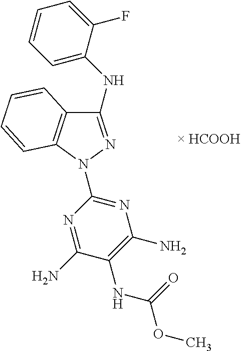 [SMILES: COC(=O)Nc1c(N)nc(-n2nc(Nc3ccccc3F)c3ccccc32)nc1N]

At 20° C., 100 mg (0.29 mmol) of 2-{3-[(2-fluorophenyl)amino]-1H-indazol-1-yl}pyrimidine-4,5,6-triamine were stirred in 1 ml of 2-propanol with 54 mg (0.4 mmol) of dimethyl dicarbonate for 18 hours. The precipitated solid was filtered off with suction and purified by preparative HPLC (Reprosil C18 10 μm, 250×30 mm, flow rate 50 ml/min, run time: 38 min, acetonitrile/water gradient+0.1% formic acid). This gave 37 mg (30% of theory) of the target compound.

LC-MS (Method 6): Rₜ=0.79 min

MS (ESIpos): m/z=409.3 (M+H)⁺

¹H-NMR (400 MHz, CDCl₃): δ=6.20 (broad s, 1H), 6.98 (m, 1H), 7.18 (dd, 1H), 7.19-7.30 (m, 2H), 7.49 (dd, 1H), 7.88 (broad s, 1H), 8.10 (d, 1H), 8.15 (s, 1H), 8.30 (dd, 1H), 8.65 (s, 1H), 8.81 (d, 1H), 12.7 (broad s, 1H).

### Example 3

### 2-{3-[(2-Fluorophenyl)sulphanyl]-1H-indazol-1-yl}pyrimidine-4,5,6-triamine

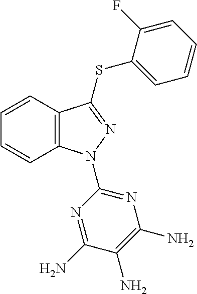 [SMILES: Nc1nc(-n2nc(Sc3ccccc3F)c3ccccc32)nc(N)c1N]

At 20° C., 3.88 g (9.76 mmol) of 2-{3-[(2-fluorophenyl)sulphanyl]-1H-indazol-1-yl}-5-nitropyrimidine-4,6-diamine were shaken with 1.559 g of 10% palladium on activated carbon in 250 ml of pyridine under a hydrogen pressure of 3.5 bar for 17 hours. The reaction was filtered and the pyridine was distilled off This gave 3.31 g (90% of theory) of the target compound.

LC-MS (Method 2): Rₜ=1.16 min

MS (ESIpos): m/z=381.2 (M+H)⁺

¹H-NMR (400 MHz, DMSO-d₆): δ=3.83 (s, 2H), 6.10 (s, 4H), 7.05-7.14 (m, 2H), 7.22-7.38 (m, 3H), 7.47-7.66 (m, 2H), 8.78 (d, 1H).

### Example 4

### 2-[3-(Pyridin-2-ylsulphanyl)-1H-indazol-1-yl]pyrimidine-4,5,6-triamine

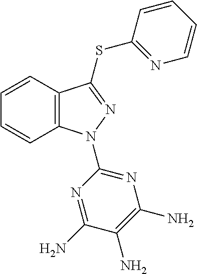 [SMILES: Nc1nc(-n2nc(Sc3ccccn3)c3ccccc32)nc(N)c1N]

At 20° C., 660 mg (1.74 mmol) of 5-nitro-2-[3-(pyridin-2-ylsulphanyl)-1H-indazol-1-yl]pyrimidine-4,6-diamine were shaken with 185 mg of 10% palladium on activated carbon in 50 ml of pyridine under a hydrogen pressure of 3.5 bar for 17 hours. Another 185 mg of 10% palladium on activated carbon were then added, and the mixture was hydrogenated under the same conditions for another 24 h. The reaction was filtered, the residue was washed with ethyl acetate and the combined filtrates were concentrated. This gave 440 mg (70% of theory) of the title compound.

LC-MS (Method 6): Rₜ=0.78 min

MS (ESIpos): m/z=351.1 (M+H)⁺

¹H-NMR (400 MHz, DMSO-d₆): δ=3.83 (broad s, 2H), 6.08 (broad s, 4H), 6.93 (d, 1H), 7.16 (m, 1H), 7.28 (dd, 1H), 7.52 (m, 2H), 7.60 (dd, 1H), 8.37 (m, 1H), 8.82 (d, 1H).

### Example 5

### 2-[3-(Pyrimidin-2-ylsulphanyl)-1H-indazol-1-yl]pyrimidine-4,5,6-triamine

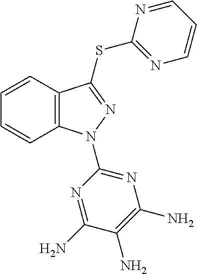 [SMILES: Nc1nc(-n2nc(Sc3ncccn3)c3ccccc32)nc(N)c1N]

At 20° C., 990 mg (2.60 mmol) of 5-nitro-2-[3-(pyrimidin-2-ylsulphanyl)-1H-indazol-1-yl]pyrimidine-4,6-diamine were shaken with 553 mg of 10% palladium on activated carbon in 75 ml of pyridine under a hydrogen pressure of 3.5 bar for 6 hours. The reaction was filtered and concentrated under reduced pressure. This gave 312 mg (9% of theory) of a solid in a purity of 27% (HPLC) which was directly reacted further without further purification.

LC-MS (Method 6): Rₜ=0.72 min

MS (ESIpos): m/z=352.0 (M+H)⁺

### Example 6

### 2-[3-(2-Fluorophenoxy)-1H-indazol-1-yl]pyrimidine-4,5,6-triamine

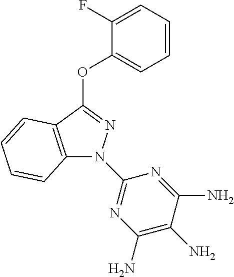 [SMILES: Nc1nc(-n2nc(Oc3ccccc3F)c3ccccc32)nc(N)c1N]

350 mg (0.92 mmol) of the compound from Example 16A were dissolved in 35 ml of pyridine, 100 mg of palladium on carbon (10%) were added and the mixture was hydrogenated under a hydrogen pressure of 3 bar for 7 h. The suspension was then filtered, the filtrat was freed from the solvent on a rotary evaporator and the residue was purified by preparative HPLC (Method 1). This gave 220 mg (68% of theory) of the target compound.

LC-MS (Method 6): Rₜ=0.84 min

MS (ESIpos): m/z=352.1 [M+H]⁺

¹H-NMR (400 MHz, DMSO-d₆): δ=3.69 (s, 2H), 4.09 (s, 4H), 7.23-7.33 (m, 3H), 7.41-7.44 (m, 2H), 7.52 (t, 1H), 7.65 (d, 1H), 8.76 (d, 1H).

### Example 8

### Methyl (4,6-diamino-2-{3-[(2-fluorophenyl)sulphanyl]-1H-pyrazolo[4,3-b]pyridin-1-yl}pyrimidin-5-yl)carbamate

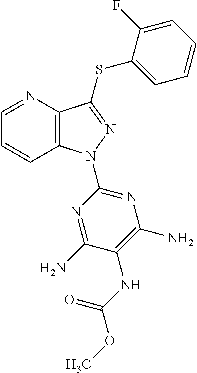 [SMILES: COC(=O)Nc1c(N)nc(-n2nc(Sc3ccccc3F)c3ncccc32)nc1N]

At 20° C., 200 mg (0.429 mmol) of 2-{3-[(2-fluorophenyl)sulphanyl]-1H-pyrazolo[4,3-b]pyridin-1-yl}pyrimidine-4,5,6-triamine (purity 79%) were stirred in 5 ml of 2-propanol and 0.5 ml of N-methylpyrrolidone with 80.51 mg (0.6 mmol) of dimethyl dicarbonate for 20 hours. The precipitated solid was filtered off with suction, washed with 5 ml of propanol and purified in DMF by preparative HPLC (Cromatorex C18 10 μm, 250×30 mm, flow rate 50 ml/min, run time: 38 min, acetonitrile/water gradient+0.1% formic acid). Evaporation of the product-containing fractions gave 76 mg (40% of theory) of the target compound.

LC-MS (Method 3): Rₜ=0.81 min

MS (ESIpos): m/z=427.3 (M+H)⁺

¹H-NMR (400 MHz, DMSO-d₆): δ=3.6 (s, 3H), 6.5 (broad s, 4H), 7.05-7.15 (m, 1H), 7.18-7.24 (m, 1H), 7.25-7.38 (m, 2H), 7.55 (dd, 1H), 7.95 (s, 1H), 8.60 (m, 1H), 9.24 (d, 1H).

### Example 9

### Propan-2-yl(4,6-diamino-2-{3-[(2-fluorophenyl)sulphanyl]-1H-pyrazolo[4,3-b]pyridin-1-yl}pyrimidin-5-yl)carbamate

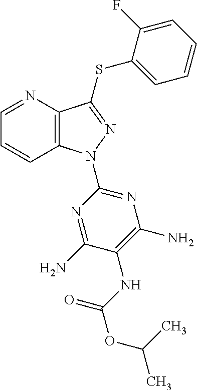 [SMILES: CC(C)OC(=O)Nc1c(N)nc(-n2nc(Sc3ccccc3F)c3ncccc32)nc1N]

At 0° C., 33.3 mg (0.271 mmol) of isopropyl chloroformate were added to 100 mg (0.214 mmol) of 2-{3-[(2-fluorophenyl)sulphanyl]-1H-pyrazolo[4,3-b]pyridin-1-yl}pyrimidine-4,5,6-triamine (purity 79%) in 5 ml of pyridine, and the mixture was stirred at 20° C. for 18 hours. After evaporation, the residue was purified by preparative HPLC (Cromatorex C18 10 μm, 250×30 mm, flow rate 50 ml/min, run time: 38 min, acetonitrile/water gradient+0.1% formic acid). Evaporation of the product-containing fractions gave 54 mg (43% of theory) of the target compound.

LC-MS (Method 3): Rₜ=0.92 min

MS (ESIpos): m/z=455.3 (M+H)⁺

¹H-NMR (400 MHz, CDCl₃): δ=1.30 (m, 6H), 5.00 (m, 1H), 5.18 (broad s, 4H), 5.68 (broad s, 1H), 6.91-7.01 (m, 1H), 7.02-7.11 (m, 1H), 7.13-7.21 (m, 2H), 7.28 (m, partially obscured by CDCl₃-Signal, 1H), 7.4 (dd, 1H), 8.60 (m, 1H), 8.97 (d, 1H).

### Example 10

### Methyl(4,6-diamino-2-{3-[(2-fluorophenyl)sulphanyl]-1H-indazol-1-yl}pyrimidin-5-yl)carbamate

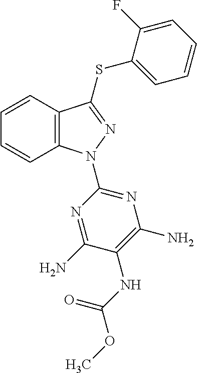 [SMILES: COC(=O)Nc1c(N)nc(-n2nc(Sc3ccccc3F)c3ccccc32)nc1N]

At 20° C., 200 mg (0.544 mmol) of 2-{3-[(2-fluorophenyl)sulphanyl]-1H-indazol-1-yl}pyrimidine-4,5,6-triamine were stirred in 6.35 ml of 2-propanol and 0.64 ml of N-methylpyrrolidone with 102.2 mg (0.76 mmol) of dimethyl dicarbonate for 19 hours. The precipitated solid was filtered off with suction and washed with 10 ml of 2-propanol. Drying under high vacuum gave 172 mg (74% of theory) of the target compound.

LC-MS (Method 6): Rₜ=0.96 min

MS (ESIpos): m/z=426.3 (M+H)⁺

¹H-NMR (400 MHz, DMSO-d₆): δ=3.6 (s, 3H), 6.43 (broad s, 4H), 7.05-7.20 (m, 2H), 7.21-7.40 (m, 3H), 7.49 (d, 1H), 7.52 (t, 1H), 7.95 (s, 1H), 8.9 (d, 1H).

### Example 11

### Methyl(4,6-diamino-2-{3-[(2-fluorophenyl)sulphanyl]-1H-indazol-1-yl}pyrimidin-5-yl)ethyl-carbamate

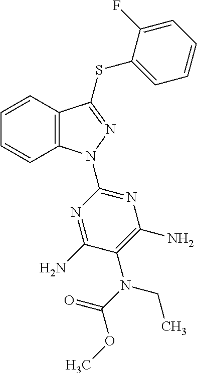 [SMILES: CCN(C(=O)OC)c1c(N)nc(-n2nc(Sc3ccccc3F)c3ccccc32)nc1N]

100 mg (0.235 mmol) of methyl (4,6-diamino-2-{3-[(2-fluorophenyl)sulphanyl]-1H-indazol-1-yl}pyrimidin-5-yl)carbamate were initially charged in 1 ml of THF, 47.4 mg (0.26 mmol) of sodium bis(trimethylsilyl)amide were added at 0° C., the mixture was stirred at 0° C. for 30 minutes, 73.3 ml (0.47 mmol) of iodoethane were added dropwise and the mixture was then stirred at 20° C. for 20 hours. After addition of 0.1 ml of water, the mixture was purified by preparative HPLC (Cromatorex C18 10 μm, 250×30 mm, flow rate 50 ml/min, run time: 38 min, acetonitrile/water gradient+0.1% formic acid). Evaporation of the product-containing fractions gave 23 mg (21% of theory) of the target compound.

LC-MS (Method 6): Rₜ=1.08 min

MS (ESIpos): m/z=454.1 (M+H)⁺

¹H-NMR (400 MHz, DMSO-d₆): δ=1.1 (t, 3H), 3.42-3.54 (m, 2H), 3.55 (s, 2H), 3.68 (s, 1H), 6.53 (s, 4H), 7.05-7.19 (m, 2H), 7.23-7.39 (m, 3H), 7.50 (d, 1H), 7.52 (dd, 1H)8.92 (d, 1H).

### Example 12

### Propan-2-yl(4,6-diamino-2-{3-[(2-fluorophenyl)sulphanyl]-1H-indazol-1-yl}pyrimidin-5-yl)-carbamate

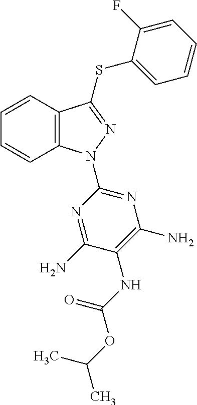 [SMILES: CC(C)OC(=O)Nc1c(N)nc(-n2nc(Sc3ccccc3F)c3ccccc32)nc1N]

At 0° C., 33.3 mg (0.271 mmol) of isopropyl chloroformate were added to 100 mg (0.272 mmol) of 2-{3-[(2-fluorophenyl)sulphanyl]-1H-indazol-1-yl}pyrimidine-4,5,6-triamine in 5 ml of pyridine, and the mixture was stirred at 20° C. for 18 hours. After evaporation, the residue was purified by preparative HPLC (Cromatorex C18 10 μm, 250×30 mm, flow rate 50 ml/min, run time: 38 min, acetonitrile/water gradient+0.1% formic acid). Evaporation of the product-containing fractions gave 109 mg (88% of theory) of the target compound.

LC-MS (Method 6): Rₜ=1.05 min

MS (ESIpos): m/z=454.3 (M+H)⁺

¹H-NMR (400 MHz, CDCl₃): δ=1.29 (d, 6H), 5.0 (qt, 1H), 5.13 (broad s, 4H), 5.68 (broad s, 1H), 6.60 (dd, 1H), 7.05 (dd, 1H), 7.10-7.22 (m, 3H), 7.40-7.53 (m, 2H), 8.71 (d, 1H).

### Example 13

### N-(4,6-Diamino-2-{3-[(2-fluorophenyl)sulphanyl]-1H-indazol-1-yl}pyrimidin-5-yl)acetamide

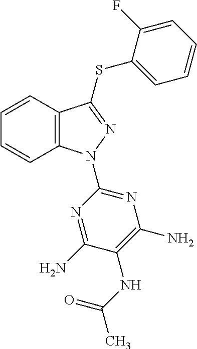 [SMILES: CC(=O)Nc1c(N)nc(-n2nc(Sc3ccccc3F)c3ccccc32)nc1N]

At 0° C., 21.3 mg (0.272 mmol) of acetyl chloride were added to 100 mg (0.272 mmol) of 2-{3-[(2-fluorophenyl)sulphanyl]-1H-indazol-1-yl}pyrimidine-4,5,6-triamine in 5 ml of pyridine, and the mixture was stirred at 20° C. for 18 hours. After evaporation, the residue was purified by preparative HPLC (Reprosil C18 10 μm, 250×30 mm, flow rate 50 ml/min, run time: 38 min, acetonitrile/water gradient+0.1% formic acid). Evaporation of the product-containing fractions gave 62 mg (56% of theory) of the target compound.

LC-MS (Method 6): Rₜ=0.92 min

MS (ESIpos): m/z=410.3 (M+H)⁺

¹H-NMR (400 MHz, CDCl₃): δ=2.02 (s, 3H), 6.35 (broad s, 4H), 7.08-7.20 (m, 2H), 7.23-7.39 (m, 3H), 7.50 (d, 1H), 7.53 (dd, 1H), 8.55 (s, 1H), 8.9 (d, 1H).

### Example 14

### N-(4,6-Diamino-2-{3-[(2-fluorophenyl)sulphanyl]-1H-indazol-1-yl}pyrimidin-5-yl)-2-methylpropanamide

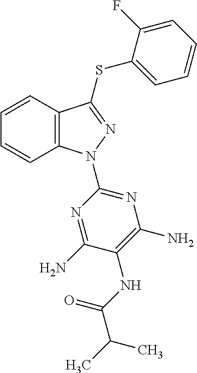 [SMILES: CC(C)C(=O)Nc1c(N)nc(-n2nc(Sc3ccccc3F)c3ccccc32)nc1N]

At 0° C., 29 mg (0.272 mmol) of 2-methylpropionyl chloride were added to 100 mg (0.272 mmol) of 2-{3-[(2-fluorophenyl)sulphanyl]-1H-indazol-1-yl}pyrimidine-4,5,6-triamine in 5 ml of pyridine, and the mixture was stirred at 20° C. for 18 hours. After evaporation, the residue was purified by preparative HPLC (Reprosil C18 10 μm, 250×30 mm, flow rate 50 ml/min, run time: 38 min, acetonitrile/water gradient+0.1% formic acid). Evaporation of the product-containing fractions gave 52 mg (43% of theory) of the target compound.

LC-MS (Method 6): Rₜ=1.00 min

MS (ESIpos): m/z=438.3 (M+H)⁺

¹H-NMR (400 MHz, CDCl₃): δ=1.12 (d, 6H), 2.62 (m, 1H), 6.25 (broad s, 4H), 7.08-7.21 (m, 2H), 7.25-7.4 (m, 3H), 7.50 (d, 1H), 7.54 (dd, 1H), 8.59 (s, 1H), 8.90 (d, 1H).

### Example 15

### N-(4,6-Diamino-2-{3-[(2-fluorophenyl)sulphanyl]-1H-indazol-1-yl}pyrimidin-5-yl)-3,3,3-trifluoropropanamide

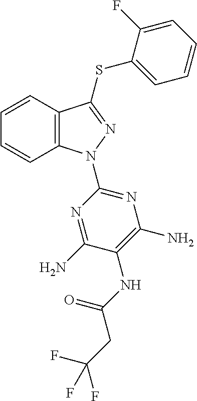 [SMILES: Nc1nc(-n2nc(Sc3ccccc3F)c3ccccc32)nc(N)c1NC(=O)CC(F)(F)F]

At 0° C., 80 mg (0.544 mmol) of 3,3,3-trifluoropropionyl chloride were added to 100 mg (0.272 mmol) of 2-{3-[(2-fluorophenyl)sulphanyl]-1H-indazol-1-yl}pyrimidine-4,5,6-triamine in 5 ml of pyridine, and the mixture was stirred at 20° C. for 42 hours. After evaporation, the residue was purified by preparative HPLC (Reprosil C18 10 μm, 250×30 mm, flow rate 50 ml/min, run time: 38 min, acetonitrile/water gradient+0.1% formic acid). Evaporation of the product-containing fractions gave 22 mg (17% of theory) of the target compound.

LC-MS (Method 6): Rₜ=1.03 min

MS (ESIpos): m/z=478.2 (M+H)⁺

¹H-NMR (400 MHz, CDCl₃): δ=3.50 (q, 2H), 6.50 (broad s, 4H), 7.09-7.20 (m, 2H), 7.28-7.40 (m, 3H), 7.50 (d, 1H), 7.56 (dd, 1H), 8.90 (d, 1H), 8.95 (s, 1H).

### Example 16

### N-(4,6-Diamino-2-{3-[(2-fluorophenyl)sulphanyl]-1H-indazol-1-yl}pyrimidin-5-yl)-3-methyl-butanamide

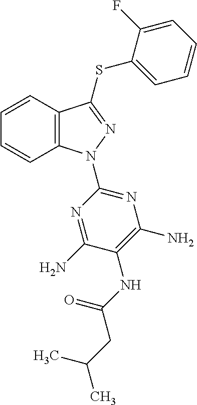 [SMILES: CC(C)CC(=O)Nc1c(N)nc(-n2nc(Sc3ccccc3F)c3ccccc32)nc1N]

At 0° C., 39 mg (0.327 mmol) of 3-methylbutyryl chloride were added to 100 mg (0.272 mmol) of 2-{3-[(2-fluorophenyl)sulphanyl]-1H-indazol-1-yl}pyrimidine-4,5,6-triamine in 5 ml of pyridine, and the mixture was stirred at 20° C. for 18 hours. After evaporation, the residue was purified by preparative HPLC (Reprosil C18 10 μm, 250×30 mm, flow rate 50 ml/min, run time: 38 min, acetonitrile/water gradient+0.1% formic acid). Evaporation of the product-containing fractions gave 58 mg (47% of theory) of the target compound.

LC-MS (Method 6): Rₜ=1.05 min

MS (ESIpos): m/z=452.3 (M+H)⁺

¹H-NMR (400 MHz, CDCl₃): δ=0.98 (d, 6H), 2.10 (m, 1H), 2.25 (d, 2H), 6.28 (broad s, 4H), 7.10-7.20 (m, 2H), 7.25-7.39 (m, 3H), 7.50 (d, 1H), 7.55 (t, 1H), 8.6 (s, 1H),8.90 (d, 1H).

### Example 17

### Methyl{4,6-diamino-2-[3-(pyridin-2-ylsulphanyl)-1H-indazol-1-yl]pyrimidin-5-yl}carbamate

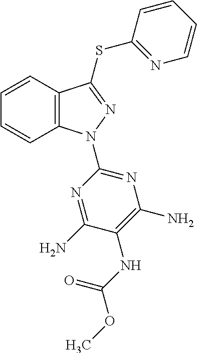 [SMILES: COC(=O)Nc1c(N)nc(-n2nc(Sc3ccccn3)c3ccccc32)nc1N]

At 20° C., 100 mg (0.285 mmol) of 2-[3-(pyridin-2-ylsulphanyl)-1H-indazol-1-yl]pyrimidine-4,5,6-triamine in 3 ml of 2-propanol and 0.3 ml of N-methylpyrrolidone were stirred with 54 mg (0.4 mmol) of dimethyl dicarbonate for 19 hours. The reaction was concentrated, 10 ml of DMF and 50 ml of water were added and the mixture was evaporated to half of its original volume. The solid which had precipitated after 16 h was filtered off with suction, washed with water and dried under high vacuum. This gave 58 mg (48% of theory) of the target compound.

LC-MS (Method 6): Rₜ=0.81 min

MS (ESIpos): m/z=409.0 (M+H)⁺

¹H-NMR (400 MHz, DMSO-d₆): δ=3.62 (s, 3H), 6.45 (s, 4H), 7.0 (d, 1H), 7.18 (m, 1H), 7.3 (t, 1H), 7.55 (m, 2H), 7.6 (dd, 1H), 7.95 (s, 1H), 8.39 (d, 1H), 8.92 (d, 1H).

### Example 18

Methyl{4,6-diamino-2-[3-(pyrimidin-2-ylsulphanyl)-1H-indazol-1-yl]pyrimidin-5-yl}carbamate

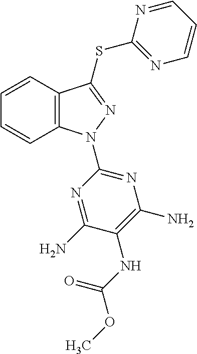 [SMILES: COC(=O)Nc1c(N)nc(-n2nc(Sc3ncccn3)c3ccccc32)nc1N]

At 20° C., 210 mg (0.077 mmol) of 2-[3-(pyrimidin-2-ylsulphanyl)-1H-indazol-1-yl]pyrimidine-4,5,6-triamine in 6.3 ml of 2-propanol and 0.63 ml of N-methylpyrrolidone were stirred with 112 mg (0.837 mmol) of dimethyl dicarbonate for 19 hours. After evaporation, the residue was purified by preparative HPLC (Cromatorex C18 10 μm, 250×30 mm, flow rate 50 ml/min, run time: 38 min, acetonitrile/water gradient+0.1% formic acid). Yield: 1 mg (0.7% of theory).

LC-MS (Method 2): Rₜ=0.83 min

MS (ESIpos): m/z=410.1 (M+H)⁺

¹H-NMR (400 MHz, DMSO-d₆): δ=3.61 (s, 3H), 6.43 (broad s, 4H), 6.72 (s, 1H), 7.28 (dd, 1H), 7.32 (dd, 1H), 7.58 (m, 2H), 7.73 (m, 1H), 8.52 (d, 2H).

### Example 19

### Propan-2-yl{4,6-diamino-2-[3-(2-fluorophenoxy)-1H-indazol-1-yl]pyrimidin-5-yl}carbamate

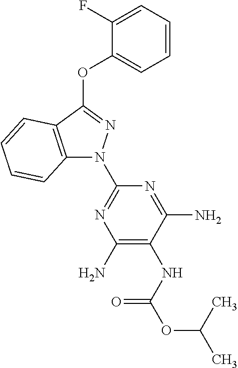 [SMILES: CC(C)OC(=O)Nc1c(N)nc(-n2nc(Oc3ccccc3F)c3ccccc32)nc1N]

80 mg (0.23 mmol) of the compound from Example 6 were dissolved in 2.8 ml of dichloromethane, 0.02 ml (0.25 mmol) of pyridine and 30.7 mg (0.25 mmol) of isopropyl chloroformate were added with ice-bath cooling and the mixture was stirred at RT for 1 h. The precipitate formed was filtered off, washed with a little dichloromethane and purified by preparative HPLC (Method 1). This gave 80 mg (80% of theory) of the target compound.

LC-MS (Method 6): Rₜ=0.96 min

MS (ESIpos): m/z (%)=438.2 (100) (M+H)⁺

¹H-NMR (400 MHz, DMSO-d₆): δ=1.17 (broad s, 6H), 4.80 (sept., 1H), 6.21 (s, 4H), 7.25-7.34 (m, 3H), 7.42-7.50 (m, 2H), 7.56 (t, 1H), 7.69 (d, 1H), 7.77 (s br, 1H), 8.88 (d, 1H).

### Example 20

Methyl{4,6-diamino-2-[3-(2-fluorophenoxy)-1H-indazol-1-yl]pyrimidin-5-yl}carbamate

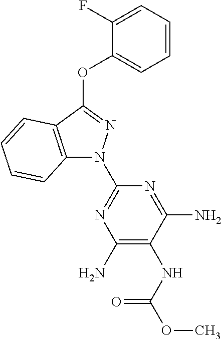 [SMILES: COC(=O)Nc1c(N)nc(-n2nc(Oc3ccccc3F)c3ccccc32)nc1N]

Analogously to the procedure of Example 19, 80 mg (0.23 mmol) of the compound from Example 6 and 23.7 mg (0.25 mmol) of methyl chloroformate gave 46 mg (49% of theory) of the target compound.

LC-MS (Method 6): Rₜ=0.89 min,

MS (ESIpos): m/z=410.2 (M+H)⁺

¹H-NMR (400 MHz, DMSO-d₆): δ=3.56 (broad s, 3H), 6.28 (s, 4H), 7.25-7.34 (m, 3H), 7.42-7.51 (m, 2H), 7.56 (t, 1H), 7.68 (d, 1H), 7.86 (s br, 1H), 8.88 (d, 1H).

### Example 21

### 2-{3-[(2-Fluorophenyl)amino]-1H-indazol-1-yl}pyrimidine-4,5,6-triamine formate

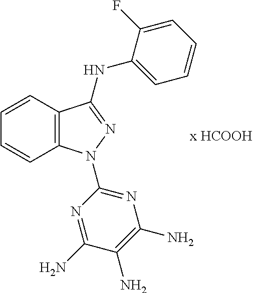 [SMILES: Nc1nc(-n2nc(Nc3ccccc3F)c3ccccc32)nc(N)c1N]

At 20° C., 630 mg (1.66 mmol) of 2-{3-[(2-fluorophenyl)amino]-1H-indazol-1-yl}-5-nitropyrimidine-4,6-diamine were shaken with 150 mg of 10% palladium on activated carbon in 147 ml of pyridine under a hydrogen pressure of 3.5 bar for 18 hours. The reaction was filtered, most of the pyridine was distilled off and the residue was purified by preparative HPLC (Reprosil C18 10 μm, 250×30 mm, flow rate 50 ml/min, run time: 38 min, acetonitrile/water gradient+0.1% formic acid). Evaporation of the product-containing fractions gave 246 mg of a solid (42% of theory).

LC-MS (Method 2): Rₜ=1.16 min

MS (ESIpos): m/z=381.2 (M+H)⁺

¹H-NMR (400 MHz, DMSO-d₆): δ=5.90 (s, 4H), 6.93 (m, 1H), 7.10-7.28 (m, 3H), 7.43 (dd, 1H), 8.03 (d, 1H), 8.12 (s, 1H), 8.26 (dd, 1H), 8.57 (s, 1H), 8.70 (d, 1H).

### Example 22

### Methyl(4,6-diamino-2-{3-[(2-fluorophenyl)sulphanyl]-1H-indazol-1-yl}pyrimidin-5-yl)(2,2,2-trifluoroethyl)carbamate

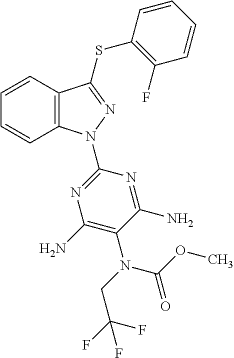 [SMILES: COC(=O)N(CC(F)(F)F)c1c(N)nc(-n2nc(Sc3ccccc3F)c3ccccc32)nc1N]

At 0° C., 100 mg (0.24 mmol) of methyl (4,6-diamino-2-{3-[(2-fluorophenyl)sulphanyl]-1H-indazol-1-yl}pyrimidin-5-yl)carbamate were stirred in 1 ml of THF with 10.3 mg (0.26 mmol) of sodium hydride for 30 minutes, 73 mg (0.26 mmol) of 2,2,2-trifluoroethyl trichloromethanesulphonate were then added and the mixture was stirred at 20° C. for 20 hours. The same amounts of sodium hydride and 2,2,2-trifluoroethyl trichloromethanesulphonate were then added once more, and the mixture was stirred at 20° C. for 2 days. After addition of 0.1 ml of water the mixture was purified by preparative HPLC (Cromatorex C18 10 μm, 250×30 mm, flow rate 50 ml/min, run time: 38 min, acetonitrile/water gradient+0.1% formic acid). This gave 20 mg (17% of theory) of the target compound.

LC-MS (Method 6): Rₜ=1.12 min

MS (ESIpos): m/z=508.0 (M+H)⁺

¹H-NMR (400 MHz, CDCl₃): δ=3.65 (s, 3H), 4.12 (m, 2H), 6.68 (broad s, 4H), 7.08-7.20 (m, 2H), 7.28-7.40 (m, 3H), 7.50 (d, 1H), 7.55 (dd, 1H), 8.93 (d, 1H).

B. Assessment of the Pharmacological Activity

The pharmacological effect of the compounds according to the invention can be shown in the following assays:

B-1. Vasorelaxant Effect In Vitro

Rabbits are stunned by a blow to the neck and exsanguinated. The aorta is removed, freed from adhering tissue and divided into rings of a width of 1.5 mm. The rings are placed individually under an initial tension in 5 ml organ baths with Krebs-Henseleit solution which is at 37° C., is gassed with carbogen and has the following composition (in each case mM): NaCl: 119; KCl: 4.8; CaCl₂×2 H₂O: 1; MgSO₄×7 H₂O: 1.4; KH₂PO₄: 1.2; NaHCO₃: 25; glucose: 10. The force of contraction is detected with Statham UC2 cells, amplified and digitized via A/D converters (DAS-1802 HC, Keithley Instruments, Munich) and recorded in parallel on chart recorders. Contractions are induced by cumulatively adding increasing concentrations of phenylephrine to the bath. After several control cycles, the substance to be investigated is added in each further run in increasing dosage, and the height of the contraction achieved is compared with the height of the contraction reached in the last preceding one. The concentration necessary to reduce the height of the control value by 50% is calculated from this (IC₅₀ value). The standard application volume is 5 μl, the proportion of DMSO in the bath solution corresponds to 0.1%.

Representative IC₅₀ values for the compounds according to the invention are shown in the table below:

| Example No. | IC₅₀ [nM] |
|---|---|
| 3 | 2145 |
| 7 | 2665 |
| 9 | 1430 |
| 12 | 4930 |
| 19 | 5640 |

B-2. Effect on a Recombinant Guanylate Cyclase Reporter Cell Line

The cellular activity of the compounds according to the invention is determined using a recombinant guanylate cyclase reporter cell line, as described in F. Wunder et al., Anal. Biochem. 339, 104-112 (2005).

B-3. Measurement of Blood Pressure on Anaesthetized Rats

Male Wistar rats having a body weight of 300-350 g are anaesthetized with thiopental (100 mg/kg i.p.). After tracheotomy, a catheter for measuring the blood pressure is introduced into the femoral artery. The substances to be tested are administered as solutions either orally by gavage or intravenously via the femoral vein (Stasch et al. Br. J. Pharmacol. 2002; 135: 344-355).

B-4. Radiotelemetric Measurement of Blood Pressure on Conscious Spontaneously Hypertensive Rats

A commercially available telemetry system from DATA SCIENCES INTERNATIONAL DSI, USA, is employed for the blood pressure measurements on conscious rats described below.

The system consists of 3 main components:

implantable transmitters (Physiotel® telemetry transmitter) receivers (Physiotel® receiver) which are linked via a multiplexer (DSI Data Exchange Matrix) to a data acquisition computer.

The telemetry system makes it possible to continuously record blood pressure, heart rate and body motions of conscious animals in their usual habitat.

Animal Material

The investigations are carried out on adult female spontaneously hypertensive rats (SHR Okamoto) with a body weight of >200 g. SHR/NCrl from the Okamoto Kyoto School of Medicine, 1963 are a cross of male Wistar Kyoto rats with highly elevated blood pressure and female rats having a slightly elevated blood pressure and at F13 handed over to the U.S. National Institutes of Health.

After transmitter implantation, the experimental animals are housed singly in type 3 Makrolon cages. They have free access to standard feed and water.

The day/night rhythm in the experimental laboratory is changed by the room lighting at 6.00 am and at 7.00 pm.

Transmitter Implantation

The telemetry transmitters TA11 PA-C40 used are surgically implanted under aseptic conditions in the experimental animals at least 14 days before the first experimental use. The animals instrumented in this way can be employed repeatedly after the wound has healed and the implant has settled.

For the implantation, the fasted animals are anaesthetized with pentobarbital (Nembutal, Sanofi, 50 mg/kg i.p.) and shaved and disinfected over a large area of their abdomens. After the abdominal cavity has been opened along the linea alba, the liquid-filled measuring catheter of the system is inserted into the descending aorta in the cranial direction above the bifurcation and fixed with tissue glue (VetBonD™, 3M). The transmitter housing is fixed intraperitoneally to the abdominal wall muscle, and layered closure of the wound is performed.

An antibiotic (Tardomyocel COMP, Bayer, 1 ml/kg s.c.) is administered postoperatively for prophylaxis of infection.

Substances and Solutions

Unless indicated otherwise, the substances to be investigated are administered orally by gavage in each case to a group of animals (n=6). The test substances are dissolved in suitable solvent mixtures, or suspended in 0.5% strength Tylose, appropriate for an administration volume of 5 ml/kg of body weight.

A solvent-treated group of animals is employed as control.

Test Procedure

The telemetry measuring unit present is configured for 24 animals. Each experiment is recorded under an experiment number (Vyear month day).

Each of the instrumented rats living in the system is assigned a separate receiving antenna (1010 Receiver, DSI).

The implanted transmitters can be activated externally by means of an incorporated magnetic switch and are switched to transmission in the run-up to the experiment. The emitted signals can be detected online by a data acquisition system (Dataquest™ A.R.T. for Windows, DSI) and be appropriately processed. The data are stored in each case in a file created for this purpose and bearing the experiment number.

In the standard procedure, the following are measured for 10-second periods in each case:
systolic blood pressure (SBP)
diastolic blood pressure (DBP)
mean arterial pressure (MAP)
heart rate (HR)
activity (ACT)

The acquisition of measured values is repeated under computer control at 5-minute intervals. The source data obtained as absolute value are corrected in the diagram with the currently measured barometric pressure (Ambient Pressure Reference Monitor; APR-1) and stored as individual data. Further technical details are given in the extensive documentation from the manufacturing company (DSI).

Unless indicated otherwise, the test substances are administered at 9.00 am on the day of the experiment. Following the administration, the parameters described above are measured over 24 hours.

Evaluation

After the end of the experiment, the acquired individual data are sorted using the analysis software (Dataquest™ A.R.T. Analysis). The blank value is assumed to be the time 2 hours before administration of the substance, so that the selected data set includes the period from 7.00 am on the day of the experiment to 9.00 am on the following day.

The data are smoothed over a presettable time by determination of the average (15-minute average) and transferred as a text file to a storage medium. The measured values presorted and compressed in this way are transferred into Excel templates and tabulated. For each day of the experiment, the data obtained are stored in a dedicated file carrying the number of the experiment. Results and test protocols are filed in paper form sorted by numbers.

Literature:

Klaus Witte, Kai Hu, Johanna Swiatek, Claudia Müssig, Georg Ertl and Björn Lemme: Experimental heart failure in rats: effects on cardiovascular circadian rhythms and on myocardial β-adrenergic signaling. Cardiovasc Res 47 (2): 203-405, 2000; Kozo Okamoto: Spontaneous hypertension in rats. Int Rev Exp Pathol 7: 227-270, 1969; Maarten van den Buuse: Circadian Rhythms of Blood Pressure, Heart Rate, and Locomotor Activity in Spontaneously Hypertensive Rats as Measured With Radio-Telemetry. Physiology & Behavior 55(4): 783-787, 1994

B-5. Determination of Pharmacokinetic Parameters After Intravenous and Oral Administration

The substance to be tested is administered intravenously as a solution to animals (for example mice, rats, dogs), and oral administration takes place as solution or suspension by gavage. After administration of the substance, blood is taken from the animals at fixed times and is heparinized, and then plasma is obtained therefrom by centrifugation. The substance is quantified analytically in the plasma by LC/MS-MS. The plasma concentration/time courses found in this way are used to calculate the pharmacokinetic parameters such as AUC, Cₘₐₓ, T_{1/2} (half-life) and CL (clearance) by means of a validated pharmacokinetic computer program.

B-6. Determination of the Solubility

Reagents Required:
PBS buffer pH 7.4: weigh 90.00 g of NaCl p.a. (for example from Merck, Cat. No. 1.06404.1000), 13.61 g of KH₂PO₄ p.a. (for example from Merck, Cat. No. 1.04873.1000) and 83.35 g of 1 N NaOH (for example from Bernd Kraft GmbH, Cat. No. 01030.4000) into a 1 litre graduated flask, make up to the mark with water and stir for about 1 hour;
acetate buffer pH 4.6: weigh 5.4 g of sodium acetate×3 H₂O, p.a. (e.g. from Merck, Cat. No. 1.06267.0500) into a 100 ml graduated flask, dissolve in 50 ml of water, add 2.4 g of glacial acetic acid, make up to 100 ml with water, check the pH and adjust to pH 4.6 if necessary;
dimethyl sulphoxide (for example from Baker, Cat. No. 7157.2500);
distilled water.

Preparation of the Calibration Solutions:

Preparation of the starting solution for calibration solutions (stock solution): About 0.5 mg of the test substance is weighed accurately into a 2 ml Eppendorf safe-lock tube (from Eppendorf, Cat. No. 0030 120.094), DMSO is added to a concentration of 600 μg/ml (e.g. 0.5 mg of substance+833 μl of DMSO), and the mixture is agitated with a vortexer until dissolution is complete.

Calibration solution 1 (20 μg/ml): 34.4 μl of the stock solution are mixed with 1000 μl of DMSO and homogenized.

Calibration solution 2 (2.5 μg/ml): 100 μl of calibration solution 1 are mixed with 700 μl of DMSO and homogenized.

Preparation of the Sample Solutions:

Sample solution for solubility up to 10 g/l in PBS buffer pH 7.4: About 5 mg of the test substance are weighed accurately into a 2 ml Eppendorf safe-lock tube (from Eppendorf, Cat. No. 0030 120.094), and PBS buffer pH 7.4 is added to a concentration of 5 g/l (e.g. 5 mg of substance+500 μl of PBS buffer pH 7.4).

Sample solution for solubility up to 10 g/l in acetate buffer pH 4.6: About 5 mg of the test substance are weighed accurately into a 2 ml Eppendorf safe-lock tube (from Eppendorf, Cat. No. 0030 120.094), and acetate buffer pH 4.6 is added to a concentration of 5 g/1 (e.g. 5 mg of substance+500 μl of acetate buffer pH 4.6).

Sample solution for solubility up to 10 g/l in water: About 5 mg of the test substance are weighed accurately into a 2 ml Eppendorf safe-lock tube (from Eppendorf, Cat. No. 0030 120.094), and water is added to a concentration of 5 g/l (e.g. 5 mg of substance+500 μl of water).

Procedure:

The sample solutions prepared in this way are shaken at 1400 rpm using a controlled-temperature shaker (e.g. Eppendorf thermomixer comfort Cat. No. 5355 000.011 with exchangeable block Cat. No. 5362.000.019) at 20° C. for 24 hours. 180 μl are removed from each of the solutions and transferred into Beckman polyallomer centrifuge tubes (Cat. No. 343621). These solutions are centrifuged at about 223 000×g for 1 hour (e.g. Beckman Optima L-90K ultracentrifuge with type 42.2 Ti rotor at 42 000 rpm). 100 μl of the supernatant are removed from each sample solution and diluted 1:5, 1:100 and 1:1000 with the solvent used in each case (water, PBS buffer 7.4 or acetate buffer pH 4.6). A portion of each dilution is dispensed into a suitable vessel for HPLC analysis.

Analysis:

The samples are analysed by RP-HPLC. A two-point calibration plot of the test compound in DMSO is used for quantification. The solubility is expressed in mg/l. Analysis sequence: 1) calibration solution 2.5 mg/ml; 2) calibration solution 20 μg/ml; 3) sample solution 1:5; 4) sample solution 1:100; 5) sample solution 1:1000.

HPLC Method for Acids:

Agilent 1100 with DAD (G1315A), quat. pump (G1311A), autosampler CTC HTS PAL, degasser (G1322A) and column thermostat (G1316A); column: Phenomenex Gemini C18, 50 mm×2 mm, 5μ; temperature: 40° C.; eluent A: water/phosphoric acid pH 2; eluent B: acetonitrile; flow rate: 0.7 ml/min; gradient: 0-0.5 min 85% A, 15% B; ramp: 0.5-3 min 10% A, 90% B; 3-3 5 min 10% A, 90% B; ramp: 3.5-4 min 85% A, 15% B; 4-5 min 85% A, 15% B.

HPLC Method for Bases:

Agilent 1100 with DAD (G1315A), quat. pump (G1311A), autosampler CTC HTS PAL, degasser (G1322A) and column thermostat (G1316A); column: VDSoptilab Kromasil 100 C18, 60 mm×2.1 mm, 3.5μ; temperature: 30° C.; eluent A: water+5 ml perchloric acid/l; eluent B: acetonitrile; flow rate: 0.75 ml/min; gradient: 0-0.5 min 98% A, 2% B; ramp: 0.5-4.5 min 10% A, 90% B; 4.5-6 min 10% A, 90% B; ramp: 6.5-6.7 min 98% A, 2% B; 6.7-7.5 min 98% A, 2% B.

C. Exemplary Embodiments of Pharmaceutical Compositions

The compounds according to the invention can be converted into pharmaceutical preparations in the following ways:

Tablet:

Composition:

100 mg of the compound according to the invention, 50 mg of lactose (monohydrate), 50 mg of corn starch (native), 10 mg of polyvinylpyrrolidone (PVP 25) (from BASF, Ludwigshafen, Germany) and 2 mg of magnesium stearate.

Tablet weight 212 mg. Diameter 8 mm, radius of curvature 12 mm

Production:

A mixture of compound according to the invention, lactose and starch is granulated with a 5% strength solution (m/m) of the PVP in water. The granules are dried and mixed with the magnesium stearate for 5 minutes. This mixture is compressed in a conventional tablet press (see above for format of the tablet). A guideline compressive force for the compression is 15 kN.

Suspension which can be Administered Orally:

Composition:

1000 mg of the compound according to the invention, 1000 mg of ethanol (96%), 400 mg of Rhodigel (xanthan gum from FMC, Pennsylvania, USA) and 99 g of water.

10 ml of oral suspension correspond to a single dose of 100 mg of the compound according to the invention.

Production:

The Rhodigel is suspended in ethanol, and the compound according to the invention is added to the suspension. The water is added while stirring. The mixture is stirred for about 6 h until the swelling of the Rhodigel is complete.

Solution which can be Administered Orally:

Composition:

500 mg of the compound according to the invention, 2.5 g of polysorbate and 97 g of polyethylene glycol 400. 20 g of oral solution correspond to a single dose of 100 mg of the compound according to the invention.

Production:

The compound according to the invention is suspended in the mixture of polyethylene glycol and polysorbate with stirring. The stirring process is continued until the compound according to the invention has completely dissolved.

i.v. Solution:

The compound according to the invention is dissolved in a concentration below the saturation solubility in a physiologically tolerated solvent (e.g. isotonic saline, 5% glucose solution and/or 30% PEG 400 solution). The solution is sterilized by filtration and used to fill sterile and pyrogen-free injection containers.

1. Compound of the formula (I)
L-A-M-Q (I),
in which
A represents O, S, —S(═O)—, S(═O)₂— or NR¹,
where
R¹ represents hydrogen or (C₁-C₄)-alkyl,
L represents (C₅-C₇)-cycloalkyl, phenyl, pyridyl, pyrimidinyl, furyl, thienyl, thiazolyl, oxazolyl, isothiazolyl or isoxazolyl,
where phenyl, pyridyl, pyrimidinyl, furyl, thienyl, thiazolyl, oxazolyl, isothiazolyl and isoxazolyl may be substituted by 1 or 2 substituents independently of one another selected from the group consisting of halogen, cyano, (C₁-C₄)-alkyl, tri-fluoromethyl, chloromethyl and (C₂-C₄)-alkynyl
and
where (C₅-C₇)-cycloalkyl may be substituted by 1 or 2 substituents independently of one another selected from the group consisting of fluorine and (C₁-C₄)-alkyl,
M represents a bicyclic heteroaryl group of the formula 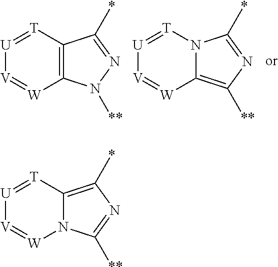
where
* represents the point of attachment to group A,
** represents the point of attachment to group Q,
T, U, V and W each represent CR² or N,
with the proviso that at most two of the ring members T, U, V and W simultaneously represent N,
and
in which
R² represents hydrogen, halogen, cyano, (C₁-C₄)-alkyl, trifluoromethyl, amino, (C₁-C₄)-alkoxy or trifluoromethoxy,
and
in which, if the substituent R² occurs more than once, its meanings may be identical or different
and
Q represents an unsaturated 5- or 6-membered heterocycle or a 5- or 6-membered heteroaryl,
where the 5- or 6-membered heterocycle and the 5- or 6-membered heteroaryl may be substituted by 1 to 4 substituents independently of one another selected from the group consisting of halogen, azido, nitro, cyano, oxo, thioxo, —R³, —C(═O)—R³, —C(═O)—OR³, —C(═O)—NR³R⁴, —O—(C═O)ₙ—R³, —O—C(═O)—OR³, —O—C(═O)—NR³R⁴, —S(O)ₚ—R³, —SO₂—OR³, —SO₂—NR³R⁴, —NR³—(C═O)ₙ—R⁴, —NR³—SO₂—R⁴, —NR³—C(═O)—OR⁴, —NR⁵—C(═O)—NR³R⁴ and —NR⁵—SO₂—NR³R⁴
in which
n represents a number 0 or 1,
p represents a number 0, 1 or 2,
R³, R⁴ and R⁵ each independently of one another represent hydrogen, (C₁-C₆)-alkyl, (C₂-C₆)-alkenyl, (C₃-C₈)-cyclo alkyl, (C₃-C₈)-cycloalkenyl, (C₆-C₁₀)-aryl, 4- to 8-membered heterocyclyl or 5- to 10-membered heteroaryl,
in which R³, R⁴ and R⁵ for their part may be substituted by 1 to 5 substituents independently of one another selected from the group consisting of halogen, azido, nitro, cyano, trifluoromethyl, (C₁-C₆)-alkyl, (C₁-C₆)-alkylcarbonyl, (C₁-C₆)-alkylcarbonyloxy, hydroxycarbonyl, (C₁-C₆)-alkoxycarbonyl, aminocarbonyl, mono-(C₁-C₆)-alkyl-aminocarbonyl, di-(C₁-C₆)-alkylaminocarbonyl, hydroxyl, trifluoromethoxy, (C₁-C₆)-alkoxy, oxo, mercapto, (C₁-C₆)-alkylthio, amino, mono-(C₁-C₆)-alkylamino, di-(C₁-C₆)-alkylamino, formylamino, (C₁-C₆)-alkylcarbonylamino, (C₁-C₆)-alkoxycarbonylamino, (C₃-C₈)-cycloalkyl, (C₃-C₈)-cycloalkenyl and 4- to 8-membered heterocyclyl,
or
R³ and R⁴ together with the radical to which the two are attached form a 4- to 8-membered heterocycle,
or
R³ and R⁵ together with the radical to which the two are attached form a 4- to 8-membered heterocycle,
or an N-oxide, salt, solvate, salt of an N-oxide or solvate of an N-oxide or salt thereof.

2. Compound of the formula (I) according to claim 1 in which
A represents O, S or NR¹,
where
R¹ represents hydrogen,
L represents phenyl, thienyl, pyridyl or pyrimidinyl,
where phenyl, thienyl, pyridyl and pyrimidinyl may be substituted by 1 or 2 substituents independently of one another selected from the group consisting of fluorine, chlorine, cyano, methyl, ethyl and trifluoromethyl,
M represents a bicyclic heteroaryl group of the formula 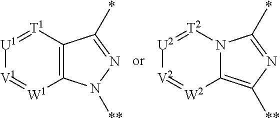
in which
* represents the point of attachment to group A,
** represents the point of attachment to group Q,
T¹, U¹, V¹ and W¹ each represent CR^{2A} or N,
where at most two of the ring members T¹, U¹, V¹ and W¹ simultaneously represent N
and
where
R^{2A} represents hydrogen or fluorine,
where at most two of the radicals R^{2A} represent fluorine
and
where, if the substituent R^{2A} occurs more than once, its meanings may be identical or different,
T², U², V² and W² each represent CR^{2B} or N,
where at most two of the ring members T², U², V² and W² simultaneously represent N
and
where
R^{2B} represents hydrogen or fluorine,
where at most two of the radicals R^{2B} represent fluorine
and
where, if the substituent R^{2B} occurs more than once, its meanings may be identical or different,
and
Q represents a group of the formula 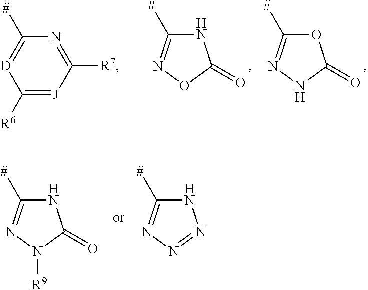
where
# represents the point of attachment to group M,
D represents CH or N,
J represents CR⁸, N or N⁺—O⁻,
in which
R⁸ represents halogen, nitro, cyano, —R³, —C(═O)—R³, —C(═O)—OR³, —C(═O)—NR³R⁴, —O—(C═O)ₙ—R³, —O—C(═O)—OR³, —O—C(═O)—NR³R⁴, —S(O)ₚ—R³, —SO₂—OR³, —SO₂—NR³R⁴, —NR³—(C═O)ₙ-R⁴, —NR³—SO₂—R⁴, —NR³—C(═O)-—OR⁴, —NR⁵—C(═O)—NR³R⁴ or —NR⁵—SO₂—NR³R⁴,
in which
n represents a number 0 or 1,
p represents a number 0 or 2,
R³, R⁴ and R⁵ each independently of one another represent hydrogen, (C₁-C₆)-alkyl, (C₂-C₆)-alkenyl, (C₃-C₇)-cycloalkyl, (C₃-C₇)-cycloalkenyl, phenyl, 5- to 7-membered heterocyclyl or 5- or 6-membered heteroaryl,
in which R³, R⁴ and R⁵ for their part may be substituted by 1 to 3 substituents independently of one another selected from the group consisting of fluorine, chlorine, cyano, (C₁-C₄)-alkyl, trifluoromethyl, hydroxyl, (C₁-C₄)-alkoxy, trifluoromethoxy, oxo, amino, mono-(C₁-C₄)-alkylamino and di-(C₁-C₄)-alkylamino,
or
R³ and R⁴ together with the radical to which the two are attached may form a 5- to 7-membered heterocycle,
or
R³ and R⁵ together with the radical to which the two are attached may form a 5- to 7-membered heterocycle,
R⁹ represents hydrogen, (C₁-C₆)-alkyl or (C₃-C₇)-cycloalkyl,
where (C₁-C₆)-alkyl may be substituted by 1 to 5 substituents independently of one another selected from the group consisting of (C₃-C₇)-cycloalkyl, hydroxyl, (C₁-C₄)-alkoxy, trifluoromethoxy, (C₁-C₄)-acyloxy, amino, mono-(C₁-C₄)-alkylamino, di-(C₁-C₄)-alkylamino, (C₁-C₄)-acylamino, hydroxycarbonyl, (C₁-C₄)-alkoxycarbonyl, aminocarbonyl, mono-(C₁-C₄)-aminocarbonyl, di-(C₁-C₄)-alkylaminocarbonyl and a 5- or 6-membered heterocycle,
or an N-oxide, salt, solvate, salt of an N-oxide or solvate of an N-oxide or salt thereof.

3. Compound of the formula (I) according to claim 1 or 2 in which
A represents S or NR¹,
where
R¹ represents hydrogen,
L represents phenyl, pyridyl or pyrimidinyl,
where phenyl may be substituted by 1 or 2 fluorine substituents,
M represents a bicyclic heteroaryl group of the formula 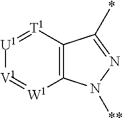
in which
* represents the point of attachment to group A,
** represents the point of attachment to group Q,
and
T¹ represents CH or N,
U¹ represents CH,
W¹ represents CH,
V¹ represents CR^{2A}
in which
R^{2A} represents hydrogen or fluorine,
Q represents a group of the formula 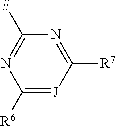
where
# represents the point of attachment to group M,
J represents CR⁸ or N
in which
R⁸ represents hydrogen, fluorine, (C₁-C₄)-alkyl, (C₃-C₇)-cycloalkyl, phenyl, pyridyl, —NR³—(C═O)ₙ—R⁴, —NR³—C(═O)—OR⁴ or —NR⁵—C(═O)—NR³R⁴
in which
n represents the number 0 or 1,
R³ represents hydrogen or (C₁-C₄)-alkyl
in which (C₁-C₄)-alkyl for its part may be substituted by a substituent selected from the group consisting of fluorine, trifluoromethyl, hydroxyl and methoxy,
R⁴ represents hydrogen, (C₁-C₄)-alkyl or (C₃-C₇)-cycloalkyl, in which (C₁-C₄)-alkyl for its part may be substituted by a substituent selected from the group consisting of fluorine, trifluoromethyl, hydroxyl and methoxy,
R⁵ represents hydrogen or (C₁-C₄)-alkyl,
or
R³ and R⁴ together with the radical to which the two are attached may form a 5- to 7-membered heterocycle,
R⁶ represents hydrogen or amino,
R⁷ represents hydrogen or amino,
or an N-oxide, salt, solvate, salt of an N-oxide or solvate of an N-oxide or salt thereof.

4. Process for preparing compounds of the formula (I), as defined in claims 1 to 3, characterized in that
[A] a compound of the formula (II) 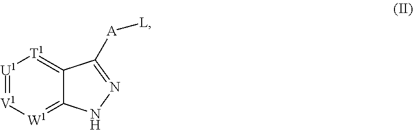
in which A, L, T¹, U¹, V¹ and W¹ each have the meanings given in any of claims 1 to 3
is reacted in an inert solvent in the presence of a palladium catalyst and a suitable base with a compound of the formula (III) 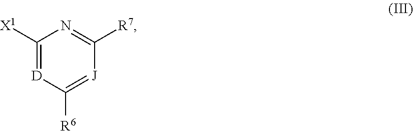
in which D, J, R⁶ and R⁷ each have the meanings given in any of claims 1 to 3 and
X¹ represents a suitable leaving group such as, for example, halogen, mesylate, tosylate or triflate
to give a compound of the formula (I-A) 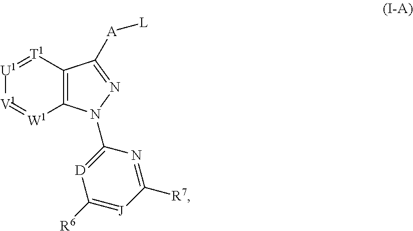
in which A, D, J, L, T¹, U¹, V¹, W¹, R⁶ and R⁷ each have the meanings given in any of claims 1 to 3,
or
[B] a compound of the formula (IV) 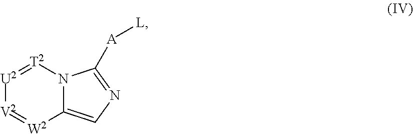
in which A, L, T², U², V² and W² each have the meanings given in any of claims 1 to 3
are, in an inert solvent, converted with a halogenating agent into a compound of the formula (V) 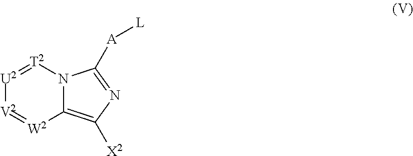
in which A, L, T², U², V² and W² each have the meanings given in any of claims 1 to 3
and
X² represents halogen, in particular bromine,
and then converted by standard methods into a tin species (VI) 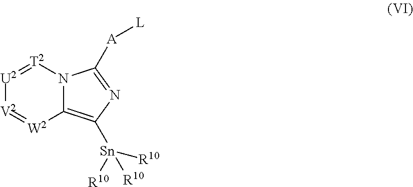
in which A, L, T², U², V² and W² each have the meanings given in any of claims 1 to 3
and
R¹⁰ represents (C₁-C₄)-alkyl,
which is then reacted in an inert solvent in the presence of a palladium catalyst and a suitable base with a compound of the formula (III)
to give a compound of the formula (I-B) 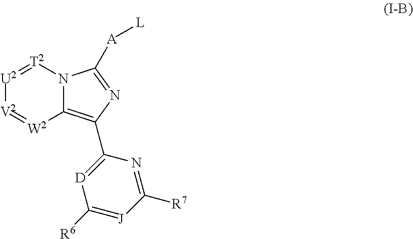
in which A, D, J, L, T², U², V², W², R⁶ and R⁷ each have the meanings given in any of claims 1 to 3,
or
[C] a compound of the formula (VII) 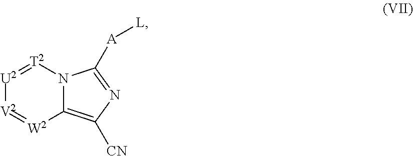
in which A, L, T², U², V² and W² each have the meanings given in any of claims 1 to 3
is reacted in an inert solvent in the presence of a suitable base with a compound of the formula (VIII) 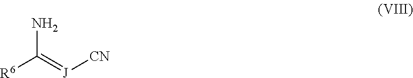
in which J and R⁶ each have the meanings given in any of claims 1 to 3,
to give a compound of the formula (I-C) 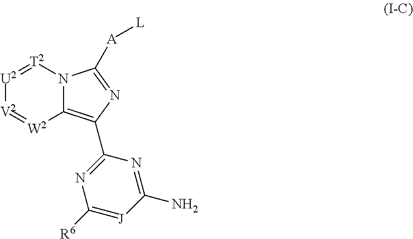
in which A, J, L, T², U², V², W² and R⁶ each have the meanings given in any of claims 1 to 3,
and the resulting compounds of the formulae (I-A), (I-B) and (I-C) are, where appropriate, converted with the appropriate (i) solvents and/or (ii) bases or acids into their solvates, salts and/or solvates of the salts.

5. Compound of the formula (I) as defined in any of claims 1 to 3 for the treatment and/or prophylaxis of diseases.

6. Compound of the formula (I) as defined in any of claims 1 to 3 for use in a method for the treatment and/or prevention of heart failure, angina pectoris, hypertension, pulmonary hypertension, ischemias, vascular disorders, thromboembolic disorders and arteriosclerosis.

7. Use of a compound of the formula (I) as defined in any of claims 1 to 3 for the preparation of a medicament for the treatment and/or prevention of heart failure, angina pectoris, hypertension, pulmonary hypertension, ischemias, vascular disorders, thromboembolic disorders and arteriosclerosis.

8. Medicament comprising a compound of the formula (I) as defined in any of claims 1 to 3 in combination with an inert, non-toxic, pharmaceutically suitable excipient.

9. Medicament comprising a compound of the formula (I) as defined in any of claims 1 to 3 in combination with a further active ingredient selected from the group consisting of organic nitrates, NO donors, cGMP-PDE inhibitors, agents having antithrombotic activity, agents lowering blood pressure, and agents altering lipid metabolism.

10. Medicament according to claim 8 or 9 for the treatment and/or prevention of heart failure, angina pectoris, hypertension, pulmonary hypertension, ischemias, vascular disorders, thromboembolic disorders and arteriosclerosis.

11. Method for the treatment and/or prevention of heart failure, angina pectoris, hypertension, pulmonary hypertension, ischemias, vascular disorders, thromboembolic disorders and arteriosclerosis in humans and animals by administration of an effective amount of at least one compound of the formula (I) as defined in any of claims 1 to 3, or of a medicament as defined in any of claims 8 to 10.
